(19) United States
(12) Patent Application Publication
(10) Pub. No.: US 2011/0112116 A1
(43) Pub. Date: May 12, 2011

(54) COMPOSITIONS OF 5-HT3 ANTAGONISTS AND DOPAMINE D2 ANTAGONISTS FOR TREATMENT OF DOPAMINE-ASSOCIATED CHRONIC CONDITIONS

(76) Inventor: NIKHILESH N. SINGH, Mill Valley, CA (US)

(21) Appl. No.: 13/009,633

(22) Filed: Jan. 19, 2011

Related U.S. Application Data
(63) Continuation of application No. 12/916,165, filed on Oct. 29, 2010.
Application No. 12/916,165 is a continuation of application No. 12/684,680, filed on Jan. 8, 2010, now abandoned.
Application No. 12/684,680 is a continuation of application No. 11/824,201, filed on Jun. 28, 2007, now abandoned.
(60) Provisional application No. 60/817,666, filed on Jun. 29, 2006.

(51) Int. Cl. A61K 31/519; A61P 25/00; A61P 25/30

(52) U.S. Cl. 514/259.41

(57) ABSTRACT

Methods are provided for partial aortic obstruction for cerebral perfusion augmentation in patients suffering from global or focal cerebral ischemia. Alternatively, the methods can be used to partially obstruct aortic blood flow to condition the spinal cord to secrete neuroprotective agents prior to abdominal aortic aneurysm repair. Partial obstruction of a vessel can be accomplished by a device comprising an elongate catheter and a distally mounted expandable member. The expandable member may comprise one or two balloons. Other medical devices, such as an angioplasty, stent, or atherectomy catheter, can be inserted distal the expandable member to provide therapeutic intervention.

### RELATED APPLICATIONS

This application is a continuation of U.S. application Ser. No. 12/916,165, filed Oct. 29, 2010, which is a continuation of U.S. application Ser. No. 12/684,680, filed Jan. 8, 2010, now abandoned, which is a continuation of U.S. application Ser. No. 11/824,201, filed Jun. 28, 2007, now abandoned, which claims the benefit of U.S. Provisional Application Ser. No. 60/817,666, entitled “Compositions and Methods for Treating Alcohol Dependence,” filed Jun. 29, 2006, all of which are hereby expressly incorporated by reference in their entirety.

### BACKGROUND OF THE INVENTION

Repetitive chronic condition or behavior is a common phenomenology of a closely related array of disorders often grouped under compulsive obsessive impulsive spectrum disorders. Even though these disorders are scattered across the fourth edition of the Diagnostic Statistical Manual (DSM IV), published by the American Psychiatric Association, they share common pathogenic features associated with aberrant sensitivity of the dopamine receptors to dopamine in the cortico-mesolimbic system of the brain. These chronic repetitive behaviors may manifest in forms that are physical (e.g., characterized by compulsive activity), mental (e.g., obsessive recurrent thoughts), psychological (e.g., substance or alcohol abuse), or pathological (e.g., excessive gambling, commission of sexual offenses, etc.).

Of the various disorders associated with chronic repetitive behaviors, alcohol abuse and dependence are recognized as major public health issues in industrialized nations. It has been estimated that in the United States alone, as much as 11 to 15 million people may be dependent on or abusing alcohol. In the U.S., alcohol dependence accounts for 9% of the intensive care unit (ICU) admissions, about 85,000 deaths per year, and costs the economy as much as $100 billion per year.

It has also been noted that physicians have an unfavorable attitude towards alcoholism and alcoholics which often impacts the treatment of the disease. Physicians' apathy towards alcoholism is surprising in view of the recognition by various National Institutes of Health organizations and the American Medical Association of alcoholism as a primary chronic disease which is often progressive and fatal. Alcohol dependence is co-morbid with a variety of psychiatric disorders such as anxiety, depression, bipolar mania, schizophrenia, etc.

Physicians often perceive alcoholism as a moral failure rather than a disease and tend. to have pessimistic view of the effectiveness of treatment interventions. Other barriers that physicians face are more practical than attitudinal. Lack of an appropriate reimbursement system for alcohol screening, assessment, and treatment, limited time and resources, lack of provider training, and an inadequate referral system all hamper as physician's efforts to help these patients.

A craving is a powerful urge to repeatedly drink and is central to abstinence from alcohol or maintenance of alcoholism. Alcohol induces craving by reinforcing the stimulatory and rewarding effects of alcohol via a neurological process that involves the interplay between 5-HT₃ receptors on the mesolimbic arm of the dopamine pathway, dopamine, and the dopamine D₂ receptor site. Alcohol or any alcohol-related cue stimulates the release of dopamine through activation of the 5-HT₃ receptors on mesolimbic dopamine neurons, which culminates in an interaction between free dopamine and the dopamine D₂ receptor on the neuron. The role of dopamine on craving and the effects of alcohol on craving are supported by studies involving dopamine D₂ antagonists such as haloperidol and tiapride. Haloperidol was reported to block increases in craving after priming doses of alcohol and tiapride effectively increased abstinence among alcoholics. These clinical findings with haloperidol and tiapride, however, are tempered due to extrapyramidal side-effects caused by simultaneous D₁ antagonist activity of the drugs.

Three drugs are currently approved in the U.S. for the treatment of alcohol dependence: disulfiram; naltrexone; and acamprosate. Disulfiram, an aldehyde dehydrogenase inhibitor, acts by interfering with the metabolic pathway of alcohol. Normally, alcohol is metabolized to acetaldehyde, which in turn is eliminated by oxidation to acetic acid by the enzyme aldehyde dehydrogenase. Disulfiram inhibits aldehyde dehydrogenase and thereby prevents oxidation of alcohol-generated acetaldehyde to acetic acid. Alcohol consumption during disulfiram treatment, however, leads to the accumulation of acetaldehyde, inducing unpleasant side-effects. Because disulfiram does not reduce craving for alcohol, success with the drug depends on a high level of patient motivation since patients who wish to drink can simply stop taking the drug. Naltrexone, a classical opiate antagonist, appears to act by reducing alcohol craving in abstinent patients; the drug, however, is hepatotoxic and causes side-effects that often require medical intervention. Acamprosate, a recently approved drug, is thought to act by modulating glutamatergic systems. It only has moderate efficacy and has yet to gain traction in the U.S.

Further it is not necessary that repetitive behavior be associated with substance or alcohol abuse. Repetitive behavior can also manifest itself in the form of recurrent or persistent thoughts, images, impulses, or mental acts performed to relieve anxiety, neutralize intrusive neurogenic signals, or relieve distress. As with alcohol and substance dependence, patients with this type of disability have a poor overall quality of life and experience significant impairment in academic functioning, work performance, and relationships. It is estimated to be the tenth leading cause of disability in the world.

Accordingly, there is a need in the art for compositions that are safe and effective for the therapeutic management of chronic repetitive behavior, e.g., alcohol dependence, on a chronic basis. The present invention satisfies this and other needs.

### SUMMARY OF THE INVENTION

The present invention provides novel compositions comprising a combination of a 5-HT₃ receptor antagonist and a selective dopamine D₂ receptor antagonist for the treatment of chronic repetitive behavior associated with abnormal sensitivity of dopamine receptors to dopamine in the eortico-mesolimbic system of the brain. The repetitive chronic conditions may be physical (e.g., characterized by a compulsive behavior), mental (e.g., obsessive thoughts), psychological (e.g., characterized by substance abuse or dependence such as alcohol dependence), or pathological (e.g., characterized by gambling, commission of sexual offenses).

Of the various disorders associated with chronic repetitive behaviors, alcohol abuse and dependence are recognized as major public health issues in industrialized nations. It has been estimated that in the United States alone, as much as 11 to 15 million people may be dependent on or abusing alcohol. In the United States, alcohol dependence accounts for 9% of the intensive care unit (ICU) admissions, about 85,000 deaths per year, and costs the economy as much as $100 billion per year. Furthermore, it is not necessary that repetitive behavior be associated with substance or alcohol abuse. Repetitive behavior can also manifest it self in the form of recurrent or persistent thoughts, images, impulses, mental acts performed to relieve anxiety, neutralize intrusive neurogenic signals or relieve distress. As with alcohol and substance dependence, patients with this type of disability have a poor overall quality of life and experience significant impairment in academic functioning, work performance, and relationships. It is estimated to be the tenth leading cause of disability in the world.

Preferably, the pharmaceutical compositions of the present invention comprise amounts of the 5-HT₃ receptor antagonist ondansetron and the selective dopamine D₂ receptor antagonist olanzapine that are sufficient to control a subject's craving for alcohol or other addictive substances. Kits comprising the combination of antagonists for the treatment of addictive disorders such as alcohol dependence are also provided.

In one aspect, the present invention provides compositions that include about 0.2 mg to about 8.0 mg of ondansetron and about 0.5 mg to about 7.5 mg of olanzapine.

In another aspect, the present invention provides methods for treating alcohol dependence in a patient. A pharmaceutical composition that includes about 0.2 mg to about 8.0 mg of ondansetron and about 0.5 mg to about 7.5 Mg olanzapine is provided. The composition is then administered to the patient.

In one aspect, the present invention provides a method for treating alcohol dependence in a subject, the method comprising administering to the subject a first amount of a 5-HT₃ receptor antagonist and a second amount of a selective dopamine d₂ receptor antagonist, wherein the amounts of the antagonists are sufficient to treat the subject.

In a related aspect, the present invention provides a method for treating a dopamine pathway-associated disease or condition in a subject, the method comprising administering to the subject a first amount of a 5-HT₃ receptor antagonist and a second amount of a selective dopamine D₂ receptor antagonist, wherein the amounts of the antagonists are sufficient to treat the subject.

In another aspect, the present invention provides a kit for the treatment of alcohol dependence comprising a first amount of a 5-HT₃ receptor antagonist and a second amount of a selective dopamine D₂ receptor antagonist, wherein the amounts of the antagonists are sufficient to treat the subject, and instructions on the use of the antagonists.

In a related aspect, the present invention provides a kit for the treatment of alcohol dependence comprising a first amount of a 5-HT₃ receptor antagonist and a second amount of a selective dopamine D₂ receptor antagonist, wherein the amounts of the antagonists are sufficient to treat the subject, and instructions on the use of the antagonists as part of a multi-step treatment program.

In another aspect, the present invention provides a kit for the treatment of repetitive chronic condition that may be physical, mental and or pathological in nature. The kit includes a first amount of a 5-HT₃ receptor antagonist and a second amount of a selective dopamine D₂ receptor antagonist, wherein the amounts of the antagonists are sufficient to treat the subject, and instructions on the use of the antagonists.

In a further aspect, the present invention provides a pharmaceutical composition comprising a first amount of a 5-HT₃ receptor antagonist and a second amount of a selective dopamine D₂ receptor antagonist.

Other objects, features, and advantages of the present invention will be apparent to one of skill in the art from the following detailed description.

### BRIEF DESCRIPTION OF THE DRAWINGS

NOT APPLICABLE

### DETAILED DESCRIPTION OF THE INVENTION

### I. General

The present invention relates to pharmaceutical compositions comprising a combination of a 5-HT₃ receptor antagonist (e.g., ondansetron) and a selective dopamine D₂ receptor antagonist, such as an atypical antipsychotic agent (e.g., olanzapine), for the therapeutic management of repetitive behavior on a chronic basis. Most of the chronic behavior conditions are largely associated with abnormal sensitivity of dopamine receptors to dopamine in the cortico-mesolimbic system of the brain. The chronic conditions may be physical (e.g., characterized by compulsive activity), mental (e.g., obsessive recurrent thoughts), psychological (e.g., characterized by substance abuse such as alcohol dependence), or pathological (e.g., characterized by gambling, commission of sexual offenses, etc.). Until now, physicians and patients have to rely largely on psychosocial therapy and drugs that are either moderately effective or have a tendency to develop tolerance. The success of these therapeutic regimens was also conditional on the patients completely abstaining from the repetitive behavior before initiation of therapy.

The present invention relates to pharmaceutical compositions comprising a combination of a 5-HT₃ receptor antagonist (e.g., ondansetron) and a selective dopamine D₂ receptor antagonist such as an atypical antipsychotic agent (e.g., olanzapine) for the therapeutic management of alcohol dependence on a chronic basis. Until now, physicians have been largely limited to psychosocial therapy and drugs for the treatment of alcohol dependence that were only moderately effective or unsafe. The success of these therapeutic regimens Was also conditional on patients completely abstaining from alcohol before and during treatment. Further, these therapeutic regimens either did not address the issue of alcohol craving or were not proven to be at all effective in reducing such craving.

The present invention overcomes these limitations by providing a combination of a 5-HT₃ receptor antagonist and a selective dopamine D₂ receptor antagonist such as an atypical antipsychotic agent in an amount sufficient to treat alcohol dependence and other drugs of abuse that results in a wider margin of safety since lower doses of each antagonist can be administered without compromising efficacy. Until now, atypical antipsychotic agents have been used to treat only psychiatric indications such as schizophrenia, acute mania, and bipolar mania. The present invention, however, demonstrates that atypical antipsychotic agents, when used in combination with 5-HT₃ receptor antagonists, are not only effective at treating dopamine aberrant conditions such as alcohol dependence and other drugs of abuse, but do so without the risk of debilitating side-effects such as sedation, metabolic syndromes such as hypoglycemia and weight gain, and extrapyramidal side-effects such as drug-induced parkinsonism. It is thought that the 5-HT₃ receptor antagonist downregulates the synthesis and/or release of dopamine, thereby augmenting the efficacy of the atypical antipsychotic agent without relapse due to neuroadaptation and without increasing the risk of extrapyramidal side-effects. Thus, the compositions of the present invention advantageously provide a safer and more effective approach for the chronic treatment and control of alcohol dependence and substance abuse.

The present invention also relates to pharmaceutical compositions comprising a combination of a 5-HT₃ receptor antagonist (e.g., ondansetron) and a selective dopamine D₂ or receptor antagonist such as an atypical antipsychotic agent (e.g., olanzapine) for the therapeutic management of chronic medical diseases or conditions associated with abnormal sensitivity to dopamine in the brain. Abnormal sensitivity to dopamine often results in conditions that reinforce recurrence of physical (e.g., characterized by compulsive behavior), psychological (e.g., characterized by substance abuse or dependence), or pathological (e.g., characterized by gambling or commission of sexual offenses) activities. Supported by several studies, the scientific and medical communities generally agree that, at a cellular level, abnormal sensitivity to dopamine is due to increased dopamine signaling caused by near-saturation occupancy of the central dopamine receptors. Behavioral reinforcement associated with these chronic medical diseases is associated with the perceived rewarding effects of the recurring activity. Even though the dopamine receptors, or more specifically, the D₂ or D₂-like receptors, are directly implicated in rewarding signals, monotherapy with dopamine D₂ or D₂-like antagonists, including atypical antipsychotics such as olanzapine, etc., have limited potential for success on a chronic basis due to counter adaptation or neuroadaptation. Neuroadaptation is an accommodative process during which the synaptic space, in an attempt to neutralize unbound dopamine, re-adjusts over time by expressing new D₂ or D₂-like receptors on the surface of the cell exposed to dopamine. Neuroadaptation often leads to development of tolerance to therapy, resulting in the patient relapsing.

The present invention overcomes these limitations by providing a safe combination of a 5-HT₃ receptor antagonist and a selective dopamine D₂ or D₂-like receptor antagonist, such as an atypical antipsychotic agent, in an amount sufficient to treat the aberrant dopamine-associated condition (without the risk of relapse) by simultaneously down-regulating the synthesis of dopamine, thereby preventing over population of dopamine in the synaptic space. The present invention demonstrates that atypical antipsychotic agents, when used in combination with 5-HT₃ receptor antagonists, are not only effective at treating medical conditions associated with abnormal sensitivity to dopamine, but do so without the risk of debilitating side-effects such as sedation, metabolic syndromes such as hypoglycemia and weight gain, and extrapyramidal side-effects such as drug-induced parkinsonism. Thus, the compositions of the present invention advantageously provide a safer and more effective approach for the chronic treatment and control of medical conditions associated with dopamine-aberrant conditions.

### II. Definitions

As used herein, the following terms have the meanings ascribed to them unless specified otherwise.

The term “alcohol use disorder” refers to a chronic disease characterized by the consumption of alcohol at a level that interferes with physical health, mental health, family responsibilities, and/or social responsibilities. There are currently two widely recognized alcohol use disorders: (I) alcoholism or alcohol dependence; and (2) alcohol abuse. “Alcoholism” or “alcohol dependence” generally refers to a disease that includes the following symptoms: craving (a strong need or urge to drink); loss of control (not being able to stop drinking once drinking has begun); physical dependence (withdrawal symptoms such as nausea, sweating, shakiness, and anxiety after stopping drinking); and tolerance (the need to drink greater amounts of alcohol to get “high”). “Alcohol abuse” typically refers to a pattern of drinking that results in one or more of the following occurring within a 12-month period; failure to fulfill major responsibilities at work, school, or home; drinking in situations that are physically dangerous; having recurring alcohol-related legal problems; and continued alcohol use despite having social or interpersonal problems caused or worsened by the effects of drinking. One skilled in the art will appreciate that alcohol use disorders can also be defined according to the formal diagnostic criteria set forth in the Diagnostic and Statistical Manual of Mental Disorders, Fourth Edition (DSM-IV), published by the American Psychiatric Association, or the International Classification Diseases, Tenth Revision (ICD-10), published by the World Health Organization.

The term “alcohol craving” as used herein refers to a conscious desire or urge to consume alcohol. Craving can occur spontaneously, or it can be elicited by internal or external stimuli (e.g., cues). Internal cues may include emotional states (e.g., anxiety) or symptoms of acute alcohol withdrawal (e.g., tremors, agitation, or seizures). External cues may include exposure to alcohol-related environments or objects (e.g., bottles of alcoholic beverages or advertisements). A subject's alcohol craving can be assessed, for example, by determining the intensity of his or her desire to drink, sometimes in the presence of an alcohol-related cue (see, e.g., Sayette et al., Addiction, 95:S189-S210 (2000)), or by detecting changes in specific physiological functions thought to accompany craving (e.g., changes in heart rate, blood pressure, salivation, or sweat gland activity) (see, e.g., Drobes et al., Alcohol Research and Health, 23:179-186 (1999)). Craving can also be assessed by directly observing a subject's drinking behavior and measuring, for example, the number of drinks consumed, the time elapsed between cue exposure and initiation of drinking (i.e., latency), and the time elapsed between commencement and completion of drinking. Additionally, a subject's alcohol craving can be assessed using a multi-item scale such as the Obsessive Compulsive Drinking Scale, which can help to assess the severity of alcoholism, monitor the progress of patients in treatment, and assess treatment outcomes (see, e.g., Anton, Addiction, 95:S211-S217, (2000); Anton et al., Archives of General Psychiatry, 53:225-231 (1996); Flannery et al., Alcoholism: Clinical and Experimental Research, 25:299-308 (2001); Roberts et al., Alcohol: Clinical and Experimental Research, 23:1484-1491 (1999)).

As used herein, the term “dopamine pathway-mediated or associated disease or condition” refers to any disease or condition that results from the abnormal sensitivity to dopamine in the mesolimbic system of the brain. Non-limiting examples of dopamine pathway-mediated diseases or conditions, which are repetitive in nature and include obsessive, impulsive and compulsive behavioral activities, schizophrenic episodes, memory loss during dementia, impairment of cognitive function, etc. The repetitive chronic conditions may be physical (e.g., characterized by a compulsive activity goal or actions performed to reduce anxiety or distress or to provide pleasure or gratification), mental obsessions (e.g., recurrent or persistent ideas, thoughts, impulses or images that are experienced as intrusive and often reinforcing), pathological (e.g., gambling, commission of sexual offenses, etc.), or psychological (e.g., characterized by substance abuse or dependence). Besides alcohol, addiction to other drugs of abuse such as nicotine (e.g., smoking, inhalation, or chewing), cocaine, amphetamine, methamphetamine, heroin, morphine, phencyclidine (PCP), methylenedioxymethamphetamine (MDMA), and opioids also result in enhanced dopamine release within the reward/reinforcement circuitry of the mesolimbic system (see, e.g., Simantov, Neurosci Lett., 163:121-124 (1993); Smith et al., Drug Discov. Today, 4:322-332 (1999); Melichar et al., Curr. Opin. Pharmacol., 1:84-90 (2001); Maldonado, J. Neural Transm. Suppl., 66:1-14 (2003)). As a result, addiction to any of these drugs can also be treated by administering the combination of antagonists described herein.

The term “antagonist” refers to a molecule which, when interacting with a biologically active molecule, blocks or negatively modulates its biological activity. Antagonists typically oppose the receptor-associated responses normally induced by other bioactive agents (i.e., agonists). Antagonists include, but are not limited to, small organic molecules, ions, proteins, nucleic acids, carbohydrates, lipids, or any other molecules that bind to or interact with biologically active molecules.

The term “selective dopamine D₂ receptor antagonist” as used herein refers to any antagonist which has selective affinity for dopamine D₂ receptors over dopamine D₁ receptors. Selective dopamine D2 receptor antagonists can also have selective affinity for dopamine D₄ receptors over dopamine D₁ receptors. Non-limiting examples of selective dopamine D₂ receptor antagonists include atypical antipsychotic agents such as olanzapine, amisulpride, aripiprazole, clozapine, fluperlapine, melperone, paliperidone, risperidone, quetiapine, sertindole, sulpiride, tiospirone, zotepine, and ziprasidone. Examples of atypical antipsychotic agents that also bind to dopamine D₄ receptors include, but are not limited to, olanzapine, clozapine, risperidone, zotepine, and tiospirone.

As used herein, the term “extrapyramidal side-effect” refers to any disorder of the extrapyramidal motor system caused by the administration of a dopamine receptor antagonist. The extrapyramidal motor system includes all of the brain structures affecting bodily (somatic) movement, e.g., the striate body (basal ganglia), its associated structures (substantia nigra, subthalamic nucleus), and its descending connections with the midbrain, but excludes the motor neurons, the motor cortex, and the pyramidal (corticobulbar and corticospinal) tract. Extrapyramidal side-effects are typically characterized by motor deficits, loss of postural reflexes, bradykinesia, tremor, rigidity, and various involuntary movements. Common extrapyramidal side-effects include, but are not limited to, akathisia (i.e., restlessness), dystonia (i.e., muscular spasms of the neck, eyes, tongue, or jaw), drug-induced parkinsonism (i.e., muscle stiffness, shuffling gait, drooling, tremor, etc.), and tardive dyskinesia (i.e., involuntary, irregular muscle movements, usually in the face). Generally speaking, atypical antipsychotic agents are associated with fewer extrapyramidal side-effects and less propensity for the development of tardive dyskinesia than typical antipsychotic agents such as haloperidol. See, e.g., Beasley et al., Neuropsychopharm., 14:111 (1996).

The term “subject” refers to humans.

The term “amount sufficient to treat the subject” refers to an amount of the combination of antagonists that is capable of achieving a therapeutic effect in a subject in need thereof. For example, an effective amount of the combination of antagonists can be the amount that is capable of diminishing or relieving one or more symptoms associated with alcohol dependence or other dopamine pathway-associated diseases or conditions.

An “immediate release component” refers to the component of a dosage form that releases the 5-HT₃ receptor antagonist and/or selective dopamine D₂ receptor antagonist within about 30 minutes (e.g., within about 30, 25, 20, 15, 14, 13, 12, 11, 10, 9, 8, 7, 6, 5, 4, 3, 2, or 1 minutes) following administration.

A “controlled release component” refers to the component of a dosage form that releases the 5-HT₃ receptor antagonist and/or selective dopamine D₂ receptor antagonist over a period of about 12 to about 48 hours (e.g., over a period of about 12, 18, 19, 20, 21, 22, 23, 24, 25, 26, 27, 28, 29, 30, 36, 42, or 48 hours) following administration.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral, transdermal, and transmucosal (e.g., buccal, sublingual, palatal, gingival, nasal, vaginal, rectal, etc.). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

The term “multi-step treatment program” refers to any program for alcohol treatment such as Alcoholics Anonymous (AA) which utilizes a series of steps that allow a subject to perform a thorough personal and emotional evaluation of what has caused him or her to abuse alcohol. Such programs typically have 12 steps, but may comprise a greater or fewer number of steps.

### III. Description of the Embodiments

In one aspect, the present invention provides a method for treating alcohol dependence in a subject, the method comprising administering to the subject a first amount of 5-HT₃ receptor antagonist and a second amount of a selective dopamine D₂ receptor antagonist, wherein the amounts of the antagonists are sufficient to treat the subject.

Examples of 5-HT₃ receptor antagonists include, but are not limited to, ondansetron, palonosetron, tropisetron, lerisetron, alosetron, granisetron, dolasetron, bernesetron, ramosetron, azaseteron, itasetron, zacopride, cilansetron, and combinations thereof. In some embodiments, the 5-HT₃ receptor antagonist is ondansetron or a pharmaceutically acceptable salt thereof.

Non-limiting examples of selective dopamine D₂ receptor antagonists include atypical antipsychotic agents such as olanzapine, amisulpride, aripiprazole, clozapine, fluperlapine, melperone, paliperidone, risperidone, quetiapine, sertindole, sulpiride, tiospirone, zotepine, ziprasidone, and combinations thereof. In some embodiments, the selective dopamine D₂ receptor antagonist is an atypical antipsychotic agent such as olanzapine or a pharmaceutically acceptable salt thereof.

In certain instances, the 5-HT₃ receptor antagonist and selective dopamine D₂ receptor antagonist are administered in combination. The antagonists can either be formulated as a single dosage form (e.g., a tablet or capsule containing both active ingredients) or as two separate dosage forms (e.g., a solid dosage form containing one active ingredient and a topical dosage form containing the other active ingredient), as long as they are administered at the same time or within about 5 minutes of each other. Alternatively, the antagonists can be administered sequentially. For example, the 5-HT₃ receptor antagonist can be administered at least about 5 minutes (e.g., at least about 5, 10, 15, 20, 25, or 30 minutes; at least about 1, 2, 3, 4, 5, 6, 7, 8, 9, 10, 15, 20, or 24 hours; or at least about 1, 2, 3, 4, 5, 6, 7, 8, 9, or 10 days) prior to or after administering the selective dopamine receptor antagonist.

In another embodiment, the combination of antagonists treats alcohol dependence by reducing the subject's alcohol craving. As a non-limiting example, a reduction in alcohol craving can be determined by assessing whether the subject has fewer drinks per day, an increase in ate number of days of abstinence, shorter episodes of alcohol consumption, and/or a longer latency between cue exposure and initiation of drinking.

In yet another embodiment, the 5-HT₃ receptor antagonist is ondansetron or a pharmaceutically acceptable salt thereof, the selective dopamine D₂ receptor antagonist is olanzapine or a pharmaceutically acceptable salt thereof, and a solid oral dose is administered wherein olanzapine is released within about 10 minutes and ondansetron is released in a controlled manner to provide systemic amounts of from about 0.25 ng/ml to about 5 ng/ml over a 24 hour period. Alternatively, the steady state plasma concentration of olanzapine over a 24 hour period may be at least 0.5 ng/ml, alternatively at least 1.5 ng/ml, alternatively no greater than 10.0 ng/ml, alternatively no greater than 20.0 ng/ml. Similarly, the steady state plasma concentration of ondansetron over a 24 hour period may be at least 0.2 ng/ml, alternatively at least 0.6 ng/ml, alternatively no greater than 1.2 ng/ml, alternatively no greater than 4.8 ng/ml.

In a related aspect, the present invention provides a method for treating a dopamine pathway-associated disease or condition in a subject, the method comprising administering to the subject a first amount of a 5-HT₃ receptor antagonist and a second amount of a selective dopamine D₂ receptor antagonist, wherein the amounts of the antagonists are sufficient to treat the subject.

Examples of dopamine pathway-mediated or associated disease or condition refers to any disease or condition that results from the abnormal sensitivity to dopamine in the mesolimbic system of the brain. Non-limiting examples of dopamine pathway-mediated diseases or conditions that are repetitive in nature and include obsessive, impulsive and compulsive behavioral activities, schizophrenic episodes, impairment of cognitive function, etc. The repetitive chronic conditions may be physical (e.g., characterized by compulsive activity goal of which may be to reduce anxiety or distress or to provide pleasure or gratification), mental obsessions (e.g., recurrent or persistent ideas, thoughts, impulses or images that are experienced as intrusive and often reinforcing), pathological (e.g., gambling, commission of sexual offenses, etc.), or psychological (e.g., (e.g., characterized by alcohol or substance abuse or dependence).

Since dopamine has been linked to addiction through its role as a pleasure chemical and appears to be the common neurotransmitter affected by all addictive substances, the combination of antagonists described herein can also be used for the therapeutic management of other types of addictions besides alcohol, such as dependence on nicotine (smoking, inhalation, chewing), cocaine, amphetamine, methamphetamine, heroin, morphine, PCP, MDMA (i.e., Ecstasy), opioids, eating, and the like. In certain instances, the combination of antagonists diminishes or relieves one or more symptoms associated with the psychological compulsive behavioral activity (e.g., reduces craving for the addictive substance). In certain other instances, the combination of antagonists stabilizes schizophrenic episodes, reduces anxiety, and improves cognitive function.

In another aspect, the present invention provides a kit for the treatment of alcohol dependence comprising a first amount of a 5-HT₃ receptor antagonist and a second amount of a selective dopamine D₂ receptor antagonist, wherein the amounts of the antagonists are sufficient to treat the subject, and instructions on the use of the antagonists.

In a related aspect, the present invention provides a kit for the treatment of alcohol dependence comprising a first amount of a 5-HT₃ receptor antagonist and a second amount of a selective dopamine D₂ receptor antagonist, wherein the amounts of the antagonists are sufficient to treat the subject, and instructions on the use of the antagonists as part of a multi-step treatment program.

In some embodiments, the 5-HT₃ receptor antagonist and selective dopamine D₂ receptor antagonist are combined in a single dosage form, e.g., in an amount effective for once-daily dosing, dosing every other day, or weekly dosing. Alternatively, the antagonists can be combined in a single dosage form in an amount effective for twice-daily dosing or dosing three, four, five, six, or more times a day. A detailed description of dosage forms suitable for use in the present invention is provided below.

In other embodiments, the 5HT₃ receptor antagonist is ondansetron or a pharmaceutically acceptable salt thereof and the selective dopamine D₂ receptor antagonist is olanzapine or a pharmaceutically acceptable salt thereof. In certain instances, the olanzapine is formulated as an immediate release component (e.g., immediate release tablet, pellet, powder, etc.) and the ondansetron is formulated as a controlled release component (e.g., prolonged release tablet, pellet, etc.).

In a further aspect, the present invention provides a pharmaceutical composition comprising a first amount of a 5-HT₃ receptor antagonist and a second amount of a selective dopamine D₂ receptor antagonist.

The pharmaceutical composition comprising the combination of antagonists is typically formulated as a depot injection, a topical dosage form, or an oral dosage form. Examples of oral dosage forms include, but are not limited to, tablets, pills, capsules, lozenges, gums, powders, solutions, suspensions, emulsions, and the like. Non-limiting examples of topical dosage forms include patches, creams, lotions, ointments, foams, aerosols, gels, oils, and the like. Suitable 5-HT₃ receptor antagonists and selective dopamine D₂ receptor antagonists are described above.

In same embodiments, the 5-HT₃ receptor antagonist is ondansetron or a pharmaceutically acceptable salt thereof and the selective dopamine D₂ receptor antagonist is olanzapine or a pharmaceutically acceptable salt thereof. In certain instances, the olanzapine is formulated as an immediate release component and the ondansetron is formulated as a controlled release component. As described in the Examples below, the immediate release component and controlled release component can be combined in a single solid dosage tablet or a single oral dosage capsule.

### A. 5-HT₃ Receptor Antagonists

A wide variety of 5-HT₃ receptor antagonists may be suitable for use in the compositions, methods, and kits described herein. For example, the 5-HT₃ receptor antagonist may be basic, acidic, or amphoteric in nature. Suitable 5-HT₃ receptor antagonists include, but are not limited to, ondansetron, palonosetron, tropisetron, lerisetron, alosetron, granisetron, dolasetron, bernesetron, ramosetron, azaseteron, itasetron, zacopride, cilansetron, and any other 5-HT₃ receptor antagonist containing imidazole, oxazole, thiazole, pyrazole, 3-pyrroline, pyrrolidine in its structural formula. The 5-HT₃ receptor antagonist downregulates the synthesis and/or release of dopamine from the mesolimbic system of the brain, either directly, indirectly, or trans-synaptically.

As used herein, the term “5-HT₃ receptor antagonist” includes all pharmaceutically acceptable forms of the 5-HT₃ receptor antagonist being described. For example, the 5-HT₃ receptor antagonist can be in a racemic or isomeric mixture, a solid complex bound to an ion exchange resin, or the like. In addition, the 5-HT₃ receptor antagonist can be in a solvated form. The term “5-HT₃ receptor antagonist” is also intended to include all pharmaceutically acceptable salts, derivatives, and analogs of the 5-HT₃ receptor antagonist being described, as well as combinations thereof. For example, the pharmaceutically acceptable salts of the 5-HT₃ receptor antagonist include, without limitation, the succinate, tartrate, bitartrate, dihydrochloride, salicylate, hemisuccinate, citrate, maleate, hydrochloride, carbamate, sulfate, nitrate, and benzoate salt forms thereof, as well as combinations thereof and the like.

Any form of the 5-HT₃ receptor antagonist is suitable for use in the present invention, e.g., a pharmaceutically acceptable salt thereof (e.g., ondansetron hydrochloride), a free base thereof, or a mixture thereof.

In general, the 5-HT₃ receptor antagonist consists of three main components: (1) an aromatic structure; (2) a carbonyl-containing linking moiety; and (3) an out-of-plane basic nitrogen containing heterocyclic group. The 5-HT₃ receptor antagonists are able retain their pharmacophore activity by either incorporating the carbonyl linker within the fused ring system, or by having the carbonyl group directly attached (as a spacer unit) to the aromatic ring and the basic nitrogen group. Those 5-HT₃ receptor antagonists belonging to the former group are exemplified by ondansetron, while those belonging to the latter group are exemplified by granisetron.

Ondansetron, which is 1, 2, 3, 9-tetrahydro-9-methyl-3-[(2-methyl-1H-imidazol-1-yl)methyl]-4H-carbazol-4-one, is described in U.S. Pat. No. 4,695,578. Ondansetron can be administered in an amount ranging from about 0.20 to about 1.0 mg, alternatively from about 0.20 to about 0.80 mg, alternatively from about 0.30 to about 0.70 mg, alternatively from about 0.325 to about 0.65 mg, alternatively from about 0.40 to about 0.60 mg. Ondansetron can also be administered in an amount ranging from about 0.50 mg to about 8.50 mg, alternatively from about 0.2 to about 8.0 mg, alternatively from about 1.0 to about 8.0 mg, alternatively from about 1.5 to about 7.5 mg, alternatively from about 2.0 to about 7.0 mg, alternatively from about 2.5 to about 7.5 mg, alternatively from about 3.0 to about 7.0 mg, alternatively from about 3.5 to about 6.5 mg, alternatively from about 4.0 to about 6.0 mg, alternatively from about 4.5 to about 5.5 mg. Alternatively, ondansetron can be administered in an amount of about 0.20 mg, about 0.25 mg, about 0.275, about 0.30 mg, about 0.325 mg, about 0.35 mg, about 0.375 mg, about 0.40 mg, about 0.425 mg, about 0.45 mg, about 0.50 mg, about 0.525 mg, about 0.55 mg, about 0.575 mg, about 0.60 mg, about 0.625 mg, about 0.65 mg, about 0.675 mg, about 0.70 mg, about 0.75 mg, about 0.80 mg, about 0.85 mg, about 0.90 mg, about 0.95 mg, about 1.00 mg, about 2.00 mg, about 3.00 mg, about 4.00 mg, about 5.00 mg, about 6.00 mg, about 7.00 mg, or about 8.00 mg. The dosages mentioned above may be used in the treatment of alcohol dependence.

Palonosetron is (3aS)-2-[(S)-1-azabicyclo[2.2.2]oct-3-yl]-2,3,3a,4,5,6-hexahydro-1-oxo-1Hbenz[de]isoquinoline and is described in U.S. Pat. No. 5,202,333. Palonsetron can be administered in an amount ranging from about 0.005 to about 0.03 mg, alternatively from about 0.005 to about 0.02 mg, alternatively from about 0.0075 to about 0.02 mg, alternatively from about 0.01 to about 0.015 mg. Alternatively, palonsetron can be administered in an amount of about 0.005 mg, about 0.0075 mg, about 0.01 mg, about 0.0125 mg, about 0.015 mg, alternatively about 0.02 mg. The dosages mentioned above may be used in the treatment of alcohol dependence.

Granisetron, which is endo-N-(9-methyl-9-azabicyclo [3.3.1] non-3-yl)-1-methyl-1H-indazole-3-carboxamide, is described in U.S. Pat. No. 4,886,808. Granisetron can be administered in an amount ranging from about 0.1 mg to about 0.5 mg, alternatively from about 0.15 to about 0.45 mg, alternatively from about 0.20 to about 0.40 mg, alternatively from about 0.25 to about 0.35 mg. Alternatively, granisetron can be administered in an amount of about 0.10 mg, about 0.15 mg, about 0.20 mg, about 0.25 mg, about 0.30 mg, about 0.35 mg, about 0.40 mg, about 0.45 mg, or about 0.50 mg. The dosages mentioned above may be used in the treatment of alcohol dependence.

Dolasetron, which is (2α,6α,8α,9αβ)-octahydro-3-oxo-2,6-methano-2H-quinolizin-8-yl-1H-indole-3-carboxylate, is described in U.S. Pat. No. 4,906,755. Dolasetron can be administered in an amount ranging from about 10 mg to about 50 mg, alternatively from about 15 mg to about 45 mg, alternatively from about 20 mg to about 40 mg, alternatively from about 25 mg to about 35 mg, alternatively from about 25 mg to about 30 mg. Alternatively, dolasetron can be administered in an amount of about 10 mg, about 15 mg, about 20 mg, about 25 mg, about 30 mg, about 35 mg, about 40 mg, about 45 mg, or about 50 mg. The dosages mentioned above may be used in the treatment of alcohol dependence.

Alosetron is 2,3,4,5-tetrahydro-5-methyl-2-[(5-methyl-1H-imidazol-4-yl)methyl]-1H-pyrido[4,3-b]indol-1-one and is described in U.S. Pat. No. 5,360,800. Alosetron can be administered in an amount ranging from about 0.05 mg to about 1.0 mg. Alternatively, alosetron can be administered in an amount from about 0.10 mg to about 0.90 mg, alternatively from about 0.20 mg to about 0.80 mg, alternatively from about 0.30 mg to about 0.70 mg, alternatively from about 0.40 mg to about 0.60 mg. The dosages mentioned above may be used in the treatment of alcohol dependence.

### B. Selective Dopamine D₂ Receptor Antagonists

A wide variety of selective dopamine D₂ receptor antagonists may be suitable for use in the compositions, methods, and kits described herein. Examples of suitable selective dopamine D₂ receptor antagonists include atypical antipsychotic agents such as those described above. Selective dopamine receptor antagonists have selectively affinity for dopamine D₂ receptors over dopamine D₁ receptors and may directly, indirectly, or trans-synaptically inhibit dopamine D₂ receptors found in the mesolimbic system of the brain. Some selective dopamine D₂ receptor antagonists also have selective affinity for dopamine D₄ receptors over dopamine D₁ receptors and inhibit dopamine D₄ receptors in the brain, either directly, indirectly, or trans-synaptically.

As used herein, the term “selective dopamine D₂ receptor antagonist” includes all pharmaceutically acceptable forms of the selective dopamine D₂ receptor antagonist being described. For example, the selective dopamine D₂ receptor antagonist can be in a racemic or isomeric mixture, a solid complex bound to an ion exchange resin, or the like. In addition, the selective dopamine D₂ receptor antagonist can be in a solvated form. The term “selective dopamine D₂ receptor antagonist” is also intended to include all pharmaceutically acceptable salts, derivatives, and analogs of the selective dopamine D₂ receptor antagonist being described, as well as combinations thereof. For example, the pharmaceutically acceptable salts of the selective dopamine D₂ receptor antagonist include, without limitation, the succinate, tartrate, bitartrate, dihydrochloride, salicylate, hemisuccinate, citrate, maleate, hydrochloride, carbamate, sulfate, nitrate, and benzoate salt forms thereof, as well as combinations thereof and the like. Any form of the selective dopamine D₂ receptor antagonist is suitable for use in the present invention, e.g., a pharmaceutically acceptable salt thereof (e.g., chlorpromazine hydrochloride), a free base thereof, or a mixture thereof.

Typical antipsychotic agents are a class of antipsychotic drugs that were first developed in the 1950s for the treatment of psychosis. Typical antipsychotic agents such as haloperidol usually have activity at both dopamine D₁ and D₂ receptors. On the other hand, atypical antipsychotic agents generally exhibit a different and recognizable clinical and pharmacological profile relative to typical antipsychotic agents. For example, atypical antipsychotic agents have dopamine D₂ receptor antagonist properties, but can also have activity at dopamine D₄ receptors and/or serotonin 5-HT₂ receptors. See, e.g., Seeman, Can. J. Psychiatry, 47:27-38 (2002); Ananth et al., J. Psychiatry Neurosci., 26:385-394 (2001). In contrast to typical antipsychotic agents, atypical antipsychotic agents are usually selective for dopamine D₂ and D₄ receptors relative to dopamine D₁ receptors. As described above, another distinguishing feature of atypical antipsychotic agents is that they are usually associated with fewer extrapyramidal side-effects and less propensity for the development of tardive dyskinesia than typical antipsychotic agents. See, e.g., Beasley et al., Neuropsychopharm., 14:111 (1996).

Olanzapine, which is 2-methyl-4-(4-methyl-1-piperazinyl)-10H-thieno-[2,3-b][1,5]benzodiazepine, is described in U.S. Pat. No. 5,229,382. Olanzapine can be administered in an amount ranging from about 0.5 mg to about 1.5 mg, alternatively from about 0.6 mg to about 1.4 mg, alternatively from about 0.7 to about 1.3 mg, alternatively from about 0.8 to about 1.2 mg, alternatively from about 0.9 to about 1.1 mg. Olanzapine can alternatively be administered in an amount ranging from about 0.5 mg to about 7.5 mg, alternatively from about mg to about 10 mg, alternatively from about 2 nig to about 9 mg, alternatively from about 3 mg to about 8 mg, alternatively from about 4 mg to about 7 mg, alternatively from about 5 mg to about 6 mg. Alternatively, olanzapine can be administered in an amount of about 0.5 mg, about 0.6 mg, about 0.7 mg, about 0.8 mg, about 0.9 mg, about 1.0 mg, about Li mg, about 1.2 mg, about 1.3 mg, about 1.4 mg, or about 1.5 mg, about 2.0 mg, about 2.5 mg, about 3.0 mg, about 3.5 mg, about 4.0 mg, about 4.5 mg, about 5.0 mg, about 5.5 mg, about 6.0 mg, about 6.5 mg, about 7.0 mg, or about 7.5 mg. The dosages mentioned above may be used in the treatment of alcohol dependence.

Risperidone is 3-[2-[4-(6-fluoro-1,2-benzisoxazol-3-yl)piperidino]e-thyl]-2-methyl-6,7,8,9-tetrahydro-4H-pyrido-[1,2-a]pyrimidin-4-one and is described in U.S. Pat. No. 4,804,663. Risperidone can be administered in an amount ranging from about 0.1 mg to about 1.0 mg, alternatively from about 0.2 mg to about 0.9 mg, alternatively from about 0.3 to about 0.8 mg, alternatively from about 0.4 to about 0.7 mg, alternatively from about 0.5 to about 1.6 mg, alternatively from about 0.1 mg to about 0.5 mg. Alternatively, risperidone can be administered in an amount of about 0.1 mg, about 0.2 mg, about 0.3 mg, about 0.4 mg, about 0.45 mg, about 0.5 mg, about 0.55 mg, about 0.6 mg, about 0.7 mg, about 0.8 mg, about 0.9 mg, or about 1.0 mg. The dosages mentioned above may be used in the treatment of alcohol dependence.

Quetiapine is 2-[2-(4-dibenzo[b,f][1,4]thiazepin-11-yl-1-piperaziny-1)ethoxy]ethanol and is described in U.S. Pat. No. 4,879,288. Quetiapine is typically administered as its (E)-2-butenedioate (2:1) salt. Quetiapine can be administered in an amount ranging from about 50 to about 150 mg, alternatively from about 60 mg to about 140 mg, alternatively from about 70 to about 1.30 mg, alternatively from about 80 to about 120 mg, alternatively from about 90 to about 110 mg. Alternatively, quetiapine can be administered in an amount of about 50 mg, about 60 mg, about 70 mg, about 80 mg, about 90 mg, about 100 mg, about 110 mg, about 120 mg, about 130 mg, about 140 mg, or about 150 mg. The dosages mentioned above may be used in the treatment of alcohol dependence.

Sertindole, which is 1-[2-[4-[5-chloro-1-(4-fluorophenyl)-1H-indol-3-yl]-1-piperidinyl]ethyl]imidazolidin-2-one, is described in U.S. Pat. No. 4,710,500. Sertindole can be administered in an amount ranging from about 1 mg to about 20 mg. Alternatively, sertindole can be administered in an amount of about 2 mg to about 18 mg, alternatively from about 4 mg to about 16 mg, alternatively from about 6 mg to about 14 mg, alternatively from about 8 mg to about 12 mg, alternatively from about 9 mg to about 11 mg. The dosages mentioned above may be used in the treatment of alcohol dependence.

Clozapine, which is 8-chloro-11-(4-methyl-1-piperazinyl)-5H-dibenzo [b,e][1,4]diazepine, is described in Hanes et al., Psychopharmacol Bull,, 24:62 (1998) and U.S. Pat. No. 3,539,573. Clozapine can be administered in an amount ranging from about 10 to about 60 mg, alternatively from about 15 mg to about 55 mg, alternatively from about 20 to about 50 mg, alternatively from about 25 to about 45 mg, alternatively from about 30 to about 40 mg. Alternatively, clozapine can be administered in an amount of about 10 mg, about 15 mg, about 20 mg, about 25 mg, about 30 mg, about 35 mg, about 45 1112, about 50 mg, about 55 mg, about 60 mg, or about 65 mg. The dosages mentioned above may be used in the treatment of alcohol dependence.

Ziprasidone is 5-[2[-4-(1,2-benzisothiazol-3-yl)piperazin-1-yl])ethyl]-6-chloro-1,3-dihydro-2H-indol-2-one hydrochloride and is described in U.S. Pat. Nos. 4,831,031, 5,312,295, 6,387,904, 6,245,765, and 6,245,766. Ziprasidone can be administered in an amount ranging from about 10 to about 100 mg, alternatively from about 2.0 mg to about 90 mg, alternatively from about 30 to about 80 mg, alternatively from about 40 to about 70 mg, alternatively from about 50 to about 60 mg. Alternatively, ziprasidone can be administered in an amount of about 10 mg, about 20 mg, about 30 mg, about 45 mg, about 50 mg, about 55 mg, about 60 mg, about 70 mg, about 80 mg, about 90 mg, or about 100 mg. The dosages mentioned above may be used in the treatment of alcohol dependence.

Amisulpride, which is 4-amino-N-[1-ethyl-2-pyrrolidinyl)methyl]-5-(-ethylsulfonyl)-2-methoxybenzamide, is described in Protais et al., Neuropharmacol., 24:861 (1985) and U.S. Pat. No. 4,401,822.

Aripiprazole is 7-[4-[4-(2,3-dichlorophenyl)piperazin-1-yl]butoxy]-3,4-dihydro-1H-quinolin-2-one. Aripiprazole can be administered in an amount ranging from about 2.0 to about 12.0 mg, alternatively from about 3.0 mg to about 11.0 mg, alternatively from about 4.0 to about 10.0 mg, alternatively from about 5.0 to about 9.0 mg, alternatively from about 6.0 to about 8.0 mg. Alternatively, aripiprazole can be administered in an amount of about 2.0 mg, about 3.0 mg, about 4.0 mg, about 5.0 mg, about 6.0 mg, about 7.0 mg, about 7.5 mg, about 8.0 mg, about 9.0 mg, about 10.0 mg, or about 11.0 mg. The dosages mentioned above may be used in the treatment of alcohol dependence.

### C. Dosage Forms

The compositions of the present invention may take the faun of solid, semi-solid, lyophilized powder, or liquid dosage forms including, for example, depot injections, topical dosage forms such as patches, creams, ointments, lotions, gels, foams, aerosols, oils, or the like, and oral dosage forms such as tablets, pills, capsules, lozenges, gums, powders, solutions, suspensions, emulsions, or the like.

As used herein, the term “dosage form” refers to physically discrete units suitable as unitary dosages for human subjects and other mammals, each unit containing a predetermined quantity of one or more active ingredients calculated to produce the desired onset, tolerability, and therapeutic effects, in association with one or more suitable pharmaceutical excipients such as carriers. Methods for preparing such dosage forms are known or will be apparent to those skilled in the art. For example, oral dosage forms can be prepared according to the procedures set forth in Remington: The Science and Practice of Pharmacy, 20th Ed., Lippincott, & Wilkins (2003); Pharmaceutical Dosage Forms, Volume 1: Tablets, 2^{nd} Ed., Marcel Dekker, Inc., New York, N.Y. (1989); and similar publications. The dosage form to be administered will, in any event, contain a quantity of each active ingredient in a therapeutically effective amount for relief of the condition being treated when administered in accordance with the teachings of this invention.

As used herein, the term “carrier” refers to a typically inert substance used as a diluent or vehicle for an active ingredient. The term also encompasses a typically inert substance that imparts cohesive qualities to the composition. Suitable carriers for use in the compositions of the present invention include, without limitation, a binder, a gum base, and combinations thereof.

Non-limiting examples of binders include mannitol, sorbitol, xylitol, maltodextrin, lactose, dextrose, sucrose, glucose, inositol, powdered sugar, molasses, starch, cellulose, microcrystalline cellulose, polyvinylpyrolidone, acacia gum, guar gum, tragacanth gum, alginate, extract of Irish moss, panwar gum, ghatti gum, mucilage of isapol husks, Veegum®, larch arabogalactan, gelatin, methylcellulose, ethylcellulose, carboxymethylcellulose, hydroxypropylmethylcellulose, polyacrylic acid (e.g., Carbopol), calcium silicate, calcium phosphate, dicalcium phosphate, calcium sulfate, kaolin, sodium chloride, polyethylene glycol, and combinations thereof. These binders can be pre-processed to improve their flowability and taste by methods known in the art such as freeze drying (see, e.g., Fundamentals of Freeze-Drying, Pharm Biotechnol., 14:281-360 (2002); Lyophililization of Unit Dose Pharmaceutical Dosage Forms, Drug. Dev. Ind. Pharm., 29:595-602 (2003)); solid-solution preparation (see, e.g., U.S. Pat. No. 6,264,987); and lubricant dusting and wet-granulation preparation with a suitable lubricating agent (see, e.g., Remington: The Science and Practice of Pharmacy, supra). For example, Mannogem® and Sorbogem®, sold by SPI Pharma Group (New Castle, Del.), are freeze-dried processed forms of mannitol and sorbitol, respectively. Typically, the compositions of the present invention comprise from about 25% to about 90% by weight of the binder, and preferably from about 50% to about 80%. However, one skilled in the art will appreciate that the compositions of the present invention can be made without any binders, e.g., to produce a highly friable dosage form.

Non-limiting examples of gum bases include materials selected from among the many water-insoluble and saliva-insoluble gum base materials known in the art. For example, in some instances, the gum base comprises at least one hydrophobic polymer and at least one hydrophilic polymer. Non-limiting examples of suitable hydrophobic and hydrophilic polymers for gum bases include both natural and synthetic polymers such as elastomers, rubbers, and combinations thereof. Examples of suitable natural polymers include, without limitation, substances of plant origin such as chicle, jelutong, gutta percha, crown gum, and combinations thereof. Examples of suitable synthetic polymers include elastomers such as butadiene-styrene copolymers, isobutylene and isoprene copolymers (e.g., “butyl rubber”), polyethylene, polyisobutylene, polyvinylester (e.g., polyvinyl acetate and polyvinyl acetate phthalate), and combinations thereof. In other instances, the gum base comprises a mixture of butyl rubber (i.e., isobutylene and isoprene copolymer), polyisobutylene, and optionally, polyvinylacetate having a molecular weight of approximately 12,000). Typically, the gum base comprises from about 25% to about 75% by weight of these polymers, and preferably from about 30% to about 60%.

The compositions of the present invention can additionally include lubricating agents; wetting agents; emulsifying agents; solubilizing agents; suspending agents; preserving agents such as methyl-, ethyl-, and propyl-hydroxy-benzoates, hutylated hydroxytoluene, and hutylated hydroxyanisole; sweetening agents; flavoring agents; coloring agents; and disintegrating agents (i.e., dissolving agents) such as crospovidone as well as croscarmellose sodium and other cross-linked cellulose polymers.

Lubricating agents can be used to prevent adhesion of the dosage form to the surface of the dies and punches, and to reduce inter-particle friction. Lubricating agents may also facilitate ejection of the dosage form from the die cavity and improve the rate of granulation flow during processing. Examples of suitable lubricating agents include, without limitation, magnesium stearate, calcium stearate, zinc stearate, stearic acid, simethicone, silicon dioxide, talc, hydrogenated vegetable oil, polyethylene glycol, mineral oil, and combinations thereof. The compositions of the present invention can comprise from about 0% to about 10% by weight of the lubricating agent, and preferably from about 1% to about 5%.

Sweetening agents can be used to improve the palatability of the composition by masking any unpleasant tastes it may have. Examples of suitable sweetening agents include, without limitation, compounds selected from the saccharide family such as the mono-, di-, tri-, poly-, and oligosaccharides; sugars such as sucrose, glucose (corn syrup), dextrose, invert sugar, fructose, maltodextrin, and polydextrose; saccharin and salts thereof such as sodium and calcium salts; cyclamic acid and salts thereof; dipeptide sweeteners; chlorinated sugar derivatives such as sucralose and dihydrochalcone; sugar alcohols such as sorbitol, sorbitol syrup, mannitol, xylitol, hexa-resorcinol, and the like, and combinations thereof. Hydrogenated starch hydrolysate, and the potassium, calcium, and sodium salts of 3,6-dihydro-6-methyl-1-1,2,3-oxathiazin-4-one-2,2-dioxide may also be used. Of the foregoing, sorbitol, mannitol, and xylitol, either alone or in combination, are preferred sweetening agents. The compositions of the present invention can comprise from about 0% to about 80% by weight of the sweetening agent, preferably from about 5% to about 75%, and more preferably from about 25% to about 50%.

Flavoring agents can also be used to improve the palatability of the composition. Examples of suitable flavoring agents include, without limitation, natural and/or synthetic (i.e., artificial) compounds such as peppermint, spearmint, wintergreen, cinnamon, menthol, cherry, strawberry, watermelon, grape, banana, peach, pineapple, apricot, pear, raspberry, lemon, grapefruit, orange, plum, apple, fruit punch, passion fruit, chocolate (e.g., white, milk, dark), vanilla, caramel, coffee, hazelnut, combinations thereof, and the like. Coloring agents can be used to color code the composition, for example, to indicate the type and dosage of the therapeutic agent therein. Suitable coloring agents include, without limitation, natural and/or artificial compounds such as FD & C coloring agents, natural juice concentrates, pigments such as titanium oxide, silicon dioxide, and zinc oxide, combinations thereof, and the like. The compositions of the present invention can comprise from about 0% to about 10% by weight of the flavoring and/or coloring agent, preferably from about 0.1% to about 5%, and more preferably from about 2% to about 3%.

Formulations suitable for oral administration include: (a) capsules, tablets, pills, or lozenges; (b) liquid solutions in a diluent such as water, saline, or PEG 400; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise each active ingredient in a flavor, sucrose, as well as pastilles comprising each active ingredient in an inert base, such as gelatin and glycerin or sucrose and acacia emulsions, gels, and the like containing, in addition to each active ingredient, carriers known in the art. Capsule forms can comprise one or more immediate release tablets, pellets, or powders and one or more prolonged release tablets or pellets, e.g., for the immediate release of a first active ingredient and the controlled release of a second active ingredient.

The immediate release tablets present in the capsule dosage forms may be prepared by direct compression of mixtures of the active ingredient or salts thereof with diluents, such as microcrystalline cellulose, mannitol, sorbitol, and lactose. Other functional excipients such as disintegrants and lubricants can be added. One of skill in the art will know how to choose the appropriate functional excipients and diluents. Alternatively, immediate release tablets may be prepared by granulation with water of a mixture of the active ingredient or salts thereof with suitable diluents, disintegrants, and binding polymers, calibration and drying of the granulate and addition of a lubricant, followed by compression on a tableting machine. The methods used are those generally described in the pharmaceutical literature; see, e.g., Sheth, Bandelin, and Shangraw, “Compressed Tablets,” in Pharmaceutical Dosage Forms: Tablets, Vol. 1, Lieberman and Lachman (Eds.), Dekker, N.Y. (1980).

The prolonged release tablets present in the capsule dosage forms can be prepared by coating immediate release tablets with a diffusion limiting polymer coating. Suitable polymers can be chosen among ethyl cellulose and methyl methacrylate copolymers such as Eudragit® RS, Eudragit® RL, and Eudragit® NE (Röhm GmbH & Co. KG; Darmstadt, Germany). Coating methods can comprise, for example, spraying a solution of the polymer on the tablets, either in a pan coater or a fluid bed coating apparatus. The solvent may be organic or aqueous, depending on the nature of the polymer used. Coating methods are known in the art and are described in, e.g., Bakan, “Microencapsulation,” in The Theory and Practice of Industrial Pharmacy, Lachman, Lieberlmman, and Kanig (Eds.), Lea & Febinger, Philadelphia (1986); and McGinity, Aqueous Polymer coatings for Pharmaceutical Dosage Forms, Dekker, N.Y. (1989). Alternatively, prolonged release tablets can be prepared by incorporating matrix-forming excipients into the formulation and omitting disintegrants. Such matrix-forming excipients may be hydrophilic polymers (e.g., hydroxypropyl methylcellulose, hydroxymethylcellulose, hydroxyethylcellulose, and the like), which swell upon contact with aqueous liquids, control the release of the active ingredient by diffusion through the swollen polymer network, and are incorporated at a level of between about 10% and about 30% by weight with respect to that of the prolonged release tablet. The matrix-forming excipient may instead be a lipidic substance, such as hydrogenated castor oil or carnuba wax, which is incorporated at a level of between about 10% and about 40% by weight with respect to that of the prolonged release tablet. In certain instances, prolonged release tablets can optionally be formulated with a pharmaceutically acceptable organic acid so as to maintain the micro-pH of the tablet during dissolution in the neutral pH conditions of the small intestine. Suitable organic acids include, but are not limited to, those comprising from 2 to 10 carbon atoms, such as lactic acid, glutamic acid, succinic acid, tartaric acid, citric acid, fumaric acid, and propionic acid. As a non-limiting example, basic 5-HT₃ receptor antagonists such as ondansetron can be formulated into prolonged release tablets with any of the above-described organic acids.

The immediate release pellets present in the capsule dosage forms may be prepared by deposition of an active ingredient onto a spherical granule, wherein the active ingredient is suspended in water or an organic solvent such as ethanol with hydroxypropyl methylcellulose or povidone or another suitable polymer to act as a binder. A fluid bed coating apparatus is generally used. Particles may be agglomerated to faun spherical granules or pellets in a high speed mixer granulator or rotary fluid bed agglomerator. These methods are known in the art and are described in, e.g., Olson et al., Int. J. Pharm. Tech. & Prod. Mfr., 6:18-24 (1985). Pellets may be also prepared by extrusion of wet masses or melts followed by spheronisation as described in, e.g., Vervaet et al., Int. J. Pharm., 116:131-146 (1995). The excipients used are typically those with plastic qualities such as microcrystalline cellulose, but can also comprise mannitol. Small quantities of a polymeric binder are generally added. Surfactants such as sodium dodecyl sulphate may also be incorporated to provide easier extrusion.

The prolonged release pellets present in the capsule dosage forms can be prepared by coating immediate release pellets in the same way as described for prolonged release tablets. Coating may be carried out, for example, in coating pans or in fluid bed coater-driers. The amount and composition of the coating is adjusted from that used in the tablet to reduce the permeability of the coating in order to take into account the far greater surface for diffusion in the pellets. In certain instances, prolonged release pellets can optionally be formulated with a pharmaceutically acceptable organic acid so as to maintain the micro-pH of the interior of the pellet during dissolution in the neutral pH conditions of the small intestine. Suitable organic acids include, but are not limited to, any of the organic acids described above, e.g., lactic acid, glutamic acid, succinic acid, tartaric acid, citric acid, fumaric acid, propionic acid, and the like. As a non-limiting example, basic selective dopamine D₂ receptor antagonists such as olanzapine can be formulated into prolonged release pellets with any of these organic acids. Alternatively, prolonged release pellets containing a basic selective dopamine D₂ receptor antagonist such as olanzapine may be coated with a pH sensitive membrane comprising at least one polymer soluble at neutral pH and impermeable at acid pH (e.g., Eudragit® S), thereby allowing increased permeation of the active ingredient at pH 5 and above, to compensate for the decreased solubility of the active ingredient at higher pH values.

When the dosage form is a tablet, the compositions of the present invention comprise a number of prolonged release coated pellets with each active ingredient embedded in a matrix. Alternatively, the tablet may comprise a mixture of prolonged release coated pellets containing a first active ingredient and immediate release non-coated pellets comprising a second active ingredient embedded in a drug-free matrix. In other embodiments, the prolonged release coated pellets containing a first active ingredient are coated with a layer comprising a second active ingredient and other excipients embedded in a drug-free matrix to allow immediate release of the second active ingredient from that layer. The matrix surrounding the pellets should preferably be formulated so that compression into tablets does not interfere with the integrity of the membrane surrounding the pellets. On contact with fluid, the tablet disintegrates and rapidly releases the first and/or second active ingredient from the matrix, the immediate release pellets, or the immediate release pellet coating, and then slowly releases the first and/or second active ingredient from the prolonged release pellets. The pellet may be formulated with a pharmaceutically acceptable organic acid so as to maintain the micro-pH of the pellet during dissolution in the neutral pH conditions of the small intestine.

In some embodiments, the tablet compositions of the present invention are in the form of a multilayer tablet comprising: (i) one or two prolonged release layers, comprising a first active ingredient and a hydrophilic polymer (e.g., a cellulose derivative); (ii) one or more immediate release layers comprising a second active ingredient; and optionally (iii) another layer not comprising any active ingredient, but comprising hydrophilic polymers such as hydroxypropylcellulose, hydroxypropylcellulose, or hydroxyethylcellulose, soluble diluents such as lactose, sorbitol, or mannitol, or hydrophilic polymers and soluble excipients, which layer modulates release of the first active ingredient from the prolonged release layer. Each layer can contain other excipients, so as to give suitable properties for compression, lubrification, and binding as is well known to one skilled in the art.

In other embodiments, the tablet compositions of the present invention are in the form of a multicoated tablet comprising: (i) a core comprising a first active ingredient such as olanzapine or ondansetron, optionally with a pharmaceutically acceptable organic acid to maintain constant pH; (ii) a polymer coating layer giving slow release of the active ingredient from the core; and (iii) a coating layer comprising a second active ingredient such as olanzapine or ondansetron, which is released rapidly or immediately on contact of the dosage form with fluid. Each portion of the tablet, in particular the inner core, can contain other excipients, so as to give suitable properties for compression, lubrification, and binding as is well known to one skilled in the art. Methods for making both multilayered and multicoated tablets are described in, e.g., Gunsel, “Compression Coated and Layer Tablets,” in Pharmaceutical Dosage Forms: Tablets, Vol. 1, Lieberman and Lachman (Eds.), Dekker, N.Y. (1980).

As further embodiments encompassed within the scope of the present invention, pharmaceutical compositions intended to avoid abuse may be included. Indeed, it is known that some drugs intended for legitimate oral use have the potential for abuse. One way of substantially reducing or even eliminating this potential for drug abuse is to provide pharmaceutical compositions for oral administration comprising olanzapine and ondansetron capable at the same time of liberating the active ingredients according to a biphasic in vitro profile following normal administration and, if it is introduced in a drink, generating a visual change in the appearance of the drink. This visual change is typically intended to avoid administration of the active ingredients to a person in a drink without his or her knowledge and includes all means of indicating the presence of the composition in a drink. The following may be used as methods for inducing visual changes: inclusion of coloring excipients, floating of the composition at the surface of the drink, formation of insoluble particles on the surface of the drink, on the brim of the glass, in the drink, and/or on the bottom of the glass, or a combination thereof.

Floating of the composition can be achieved by an effervescence which can be obtained by means of an effervescence generator. In addition to these effervescent properties, the composition can present viscosity increasing properties appearing on contact with the drink. Thus, when the bubbles are formed, they are “trapped” and the composition swells. The lowering of the density contributes to maintaining the pharmaceutical composition at the surface of the drink. Such a viscosity may be obtained by one or more gelating substances. Hydrophilic excipients are particularly suitable as gel-forming substances as set forth herein. Particles may be obtained by association of a lipophilic and a hydrophilic excipient, useful for the floating of the composition as described above. A list of suitable lipophilic excipients is set forth herein. The composition according to this particular embodiment of the present invention can liberate particles even if the composition does not float or not immediately.

The effervescence generator can be a carbon dioxide generator system comprising a suitable carbon dioxide generator agent and a pharmaceutically acceptable acid. The carbon dioxide generator agent is usually a carbonate or bicarbonate of an alkali or alkaline earth metal or an amino acid. Calcium carbonate, sodium bicarbonate, potassium carbonate, potassium bicarbonate, L-lysine carbonate, arginine carbonate, or sodium sesquicarbonate may be used as carbon dioxide generator agents. The acid may be an acid anhydride, a monocarboxylic acid, a polycarboxylic acid, or a partial salt of a polycarboxylic acid. More particularly, citric, tartric, ascorbic, fumaric, nicotinic, acetysalicylic, maleic, adipic, succinic, malic, or malonic acid may be chosen or glutaric anhydride, citric anhydride, monosodium citrate, or succinic anhydride. In certain instances, the carbon dioxide generator agent comprises a mixture of carbon dioxide generating agents described above. The content of the acidic compound is generally chosen such that the ratio between the number of moles in the acidic compound with respect to the number of moles in the carbon dioxide generator agent is between about 1 and about 2.

The gel forming substance can comprise one or more hydrophilic excipients provoking the swelling of the composition and the trapping of the gas released. In order to form insoluble particles, one or more lipophilic excipients are added to the hydrophilic excipient. The process of effervescence and formation of particles generates viscous agglomerates which float and stick to the glass. This process can last between about 0.5 and about 25 minutes, depending on the type of drink. Lipophilic excipients suitable for use include, but are not limited to, glycerol stearates, palmitostearates, and behenates, hydrogenated vegetable oils and their derivatives, vegetable and animal wax and their derivatives, hydrogenated castor oils and their derivatives, and cetylic esters and alcohols. Hydrophilic excipients that may be used include, for example, cellulose derivatives, hydroxyethylcellulose, hydroxypropylcellulose (molecular mass from 50 to 1250 kDa), hydroxypropyl methylcellulose (molecular mass from 10 to 1500 kDa), carboxymethylcellulose, and sodium carboxymethylcellulose, vegetable gums and their derivatives, derivatives of alginic acid, polyethyleneglycols and their derivatives, starches and their derivatives, silica, polymethacrylates, acrylic acid, and methacrylate copolymers. In certain instances, one of the constituents of the gel forming substance can be chosen as being less soluble in alcohol.

A coloring excipient can be advantageously added as giving rise to a visual change preventing abuse. It can color simultaneously the liquid or the particles or one independently of the other. Among suitable coloring excipients include, without limitation, indigotine, cochineal carminic acid, yellow orange S, allura red AC, iron oxides, cucurmin, riboflavin, tartrazine, quinoline yellow, azorubine, amaranth, carmines, erythosine, red 2G, patented blue V, glittering blue FCF, chlorophylls, copper complexes of chlorophylls, green S, caramel, glittering black BN, carbo medicinalis vegetabilis, brown FK and HT, carotenoids, Annatto extracts, paprika extracts, lycopene, lutein, canthaxanthin, beetroot red, anthocyanes, calcium carbonate, titanium dioxide, aluminum, silver, gold or litholrubin BK, and any other coloring excipient suitable for oral administration. These visual means of preventing abuse may comprise a distinct pharmaceutical component, not containing any active ingredients, along with the immediate release and the sustained release components, that comprise the pharmaceutical form, or they may be incorporated in one of these two components. Yet a third method is to incorporate most or all of the visual means into a separate component and at the same time add some to the immediate and/or sustained release components.

The method of incorporation of abuse resistance as described above will depend on the type of formulation. In the case of tablet formulations, including that of tablets enclosed inside a capsule, the abuse resistance-conferring substances (e.g., coloring matter, effervescent couple, etc.) may be included within the immediate release component of the formulation. Alternatively, in the case of multilayer tablets and immediate release tablets within a capsule, they may be incorporated as a separate layer not containing active ingredients, but with the abuse resistance-conferring substances. Such a layer may be added to the sustained release tablet or tablets within a capsule provided that the tablet is formulated as a matrix and is not coated with a coating conferring the sustained release properties. In the case of a capsule containing controlled release pellets and immediate release pellets or granules, abuse resistance-conferring substances, with the exception of an effervescent couple, may be incorporated in the immediate release component or added separately.

The compositions of the present invention can also be made into any form suitable for topical administration. As a non-limiting example, one or more active ingredients can be delivered by a transdermal delivery system (i.e., a patch) comprising a backing layer and an adhesive polymer matrix which has dispersed therein the one or more active ingredients, skin permeation enhancers, and a plasticizer/humectant. Preferably, the transdermal delivery system comprises a combination of 5-HT₃ receptor and selective dopamine D receptor antagonists in an amount sufficient for treating a dopamine pathway-associated disease or condition such as alcohol dependence.

The backing layer of the transdermal delivery system can be made of any suitable material, which is impermeable to the one or more active ingredients dispersed within the adhesive polymer matrix. The backing layer serves as a protective cover for the matrix layer and also provides a support function. The backing layer can be formed so that it is essentially the same size as the drug-containing adhesive polymer matrix. Alternatively, the backing layer can be of a larger dimension so that it can extend beyond the side of the adhesive polymer matrix or overlay the side or sides of the adhesive polymer matrix and then can extend outwardly in a manner such that the surface of the extension of the backing layer can be the base for an adhesive means. For long-term applications, e.g., for seven or more days, it may be desirable to use microporous and/or breathable backing laminates, so hydration or maceration of the skin can be minimized.

Examples of materials suitable for making the backing layer include, but are not limited to, films of high and low density polyethylene, polyproplene, polyurethane, polyvinylchloride, polyesters such as polyethylene phthalate), metal foils, metal foil Laminates of such suitable polymer films, and the like. Preferably, the materials used for the backing layer are laminates of such polymer films with a metal foil such as aluminum foil. In such laminates, a polymer film of the laminate will usually be in contact with the adhesive. polymer matrix.

The backing layer can be any appropriate thickness which will provide the desired protective and support functions. A suitable thickness can be from about 10 to about 300 microns, alternatively from about 15 to about 20 microns, or alternatively from about 30 to about 100 microns.

Generally, the polymers used to form the biologically acceptable adhesive polymer layer are those capable of forming thin films or coatings through which the one or more active ingredients can pass at a controlled rate. Suitable polymers are biologically and pharmaceutically compatible, non-allergenic, and insoluble in and compatible with body fluids or tissues with which the device is contacted. The use of soluble polymers should be avoided since dissolution or erosion of the matrix would affect the release rate of the one or more active ingredients as well as the capability of the dosage unit to remain in place for convenience of removal.

Exemplary materials for fabricating the adhesive polymer layer include, without limitation, polyethylene, polypropylene, ethylene/propylene copolymers, ethylenelethylacrylate copolymers, ethylene/vinyl acetate copolymers, silicone elastomers (e.g., medical-grade polydimethylsiloxanes), neoprene rubber, polyisobutylene, polyacrylates, chlorinated, polyethylene, polyvinyl chloride, vinyl chloride-vinyl acetate copolymers, crosslinked polymethacrylate polymers (e.g., hydro-gels), polyvinylidene chloride, polyethylene terephthalate), butyl rubber, epichlorohydrin rubbers, ethylenvinyl alcohol copolymers, ethylene-vinyloxyethanol copolymers; silicone copolymers (e.g., polysiloxane-polycarbonate copolymers, polysiloxanepolyethylene oxide copolymers, polysiloxane-polymethacrylate copolymers, polysiloxane-alkylene copolymers such as polysiloxane-ethylensilane copolymers, and the like), cellulose polymers (e.g., methyl or ethyl cellulose, hydroxypropyl methy cellulose, and cellulose esters), polycarbonates, polytetrafluoroethylene, and mixtures thereof.

The biologically acceptable adhesive polymer matrix is typically selected from polymers with glass transition temperatures below room temperature. The polymer may, but need not necessarily, have a degree of crystallinity at room temperature. Cross-linking monomeric units or sites can be incorporated into such polymers. Non-limiting examples of cross-linking monomeric units include polymethacrylic estes of polyols such as butylene diacrylate and dimethacrylate, trimethylol propane trimethacrylate, and the like. Other monomers which provide such sites include allyl acrylate, allyl methacrylate, diallyl maleate, and the like.

Preferably, the adhesive polymer matrix comprises a polyacrylate adhesive polymer such as, e.g., a polyacrylate adhesive copolymer comprising a 2-ethylhexyl acrylate monomer and about 50%-60% w/w of vinyl acetate as a co-monomer. An example of a suitable polyacrylate adhesive copolymer for use in the present invention includes, but is not limited to, DURO-TAK® 87-4098 by National Starch and Chemical Co. (Bridgewater, N.J.), which comprises a certain percentage of vinyl acetate co-monomer.

The specific active ingredients which may be dispersed in the adhesive polymer matrix include any combination of 5-HT₃ receptor and selective dopamine D₂ receptor antagonists capable of treating a dopamine pathway-associated disease or condition and of being transdermally administered. With the controlled release of the antagonists at a relatively steady rate over a prolonged period, typically several days or one week, the subject is provided with the benefit of a steady infusion of sufficient amounts of the antagonists over a prolonged period.

It is presently preferred to transdermally deliver olanzapine and ondansetron by an adaptable system described herein at a desirable daily rate. For example, olanzapine and ondansetron can dispersed in the matrix layer-forming polymer. Generally, a transdermal dosage unit designed for one-week therapy is required to deliver at least about 2.5 mg/day olanzapine or an equivalent effective amount of a selective dopamine D₂ receptor antagonist, and about 4 mg/day ondansetron or an equivalent effective amount of a 5-HT₃ receptor antagonist.

In some embodiments, a plasticizer/humectant or permeability enhancer is dispersed within the adhesive polymer matrix. The plasticizer/humectant may be a conventional plasticizer used in the pharmaceutical industry, for example, polyvinyl pyrrolidone (PVP). As a non-limiting example, PVP/vinyl acetate (VA), such as those having a molecular weight of from about 50,000, can be used with the present invention. The PVP/VA acts both as a plasticizer, to control the rigidity of the polymer matrix, as well as a humectant, to regulate the moisture content of the formulation. Incorporation of a humectant in the formulation allows the dosage unit to absorb moisture on the surface of the skin, which in turn helps to reduce skin irritation and to prevent the adhesive polymer layer of the delivery system from failing. In certain instances, the plasticizer and/or humectant is PVP/VA S-630 from ISP International Specialty Products, Inc. (Wayne, N.J.), wherein the PVP is present in an amount of about 60% by weight and the VA is present in an amount of about 50% by weight of the total mixture.

Depending upon the antagonists used and the drug delivery desired, a suitable amount of a plasticizer can range from about 0% to about 10% by weight based on the weight of the adhesive polymer matrix. Preferably, the amount of humectant/plasticizer used is less than about 5%.

Drug molecules released from a transdermal delivery system must be capable of penetrating each layer of skin. In order to increase the rate of permeation of drug molecules, a transdermal drug delivery system must be able to increase the permeability of the outermost layer of skin, the stratum corneum, which provides the most resistance to the penetration of drug molecules. In certain instances, a combination of skin permeation enhancing agents comprising a mixture of dimethyl sulfoxide (DMSO), a fatty alcohol ester of lactic acid such as lauryl lactate (Ceraphil 31), a lower alkanol ester of lactic acid such as ethyl lactate, and capric acid is employed in the practice of the present invention. It is further preferred that these skin permeation enhancers be present at a weight ratio of from about 2.0:1:1:0.8 to about 6:1:1:0.8, or alternatively about 4:1:1:0.8. The total amount of enhancer mixture can be about 10% to about 60% w/w of the polymer matrix, preferably about 43% w/w when an acrylate copolymer is used.

In making the adhesive polymer matrix, polyacrylate adhesive polymers such as those described above are preferably used. The antagonists can be added in an amount determined by the antagonist dosage and the duration of treatment desired in each dosage unit. It has been found, for example, that one part total of antagonists can be satisfactorily added to about 75 parts of the polyacrylate adhesive polymer used in making the polymer matrix.

Prior to mixing with a polyacrylate adhesive polymer, the antagonists used are typically dissolved and dispersed in a solution comprising a PVP/VA and a combination of skin permeation enhancers. In certain instances, the enhancer combination and the plasticizer solution are combined, and the antagonists added thereto and subjected to mixing. The amount of enhancers used depends in part on the rapidity at which the antagonists are to be delivered. Generally speaking, about 10% to about 60% of the skin permeation enhancer combination based on the weight of the adhesive polymer matrix solution is suitable. Preferably, about 40% to about 45% of the skin permeation enhancer combination is used. It is also preferred that the drug-containing adhesive polymer matrix contain some excess of the dispersed antagonists over the dosage amount desired to be delivered thereby. For example, the excess can be about 5 to about 50 times the desired dosage, or alternatively, about 10 to about 25 times the desired dosage to be transdermally absorbed.

The adhesive polymer solution can then be added to the solution of antagonists dispersed in the enhancer combination/plasticizer solution. The mixture of the polyacrylate adhesive copolymer and the enhancer/plasticizer/antagonist solution is then thoroughly mixed using a high-torque mixer to form a homogeneous dispersion or solution of the antagonists in the polyacrylate adhesive copolymer. The composition can then be allowed to stand undisturbed until deaerated, i.e., for a time period of at least one hour up to about 24 hours.

Once deaerated, the adhesive polymer matrix is preferably applied to a backing layer material, such as, for example, Scotch Pak 1109 from 3M Co, (St. Paul, Minn.) and subsequently dried at 60° C. for about 15 minutes. The dried adhesive polymer matrix can then be laminated with a piece of release liner (e.g., Scotch Pak 1012 from 3M Co.),

The compositions of the present invention, either alone or in combination with other suitable components, can also be made into aerosol formulations (i.e., they can be “nebulized”) to be administered via inhalation (e.g., intranasally intratracheally) (see, e.g., Brigham et al., Am. J. Sci., 298:278 (1989)). Aerosol formulations can be placed into pressurized acceptable propellants, such as dichlorodifluoromethane, propane, nitrogen, and the like.

Formulations suitable for parenteral administration, such as, for example, by intravenous, intraarticular (in the joints), intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Generally, when administered intravenously, the formulations of the present invention are formulated with a suitable pharmaceutical carrier. A variety of aqueous carriers may be used, for example, water, buffered water, 0.4% saline, 0.3% glycine, and the like, and may include glycoproteins for enhanced stability, such as albumin, lipoprotein, globulin, etc. Generally, normal buffered saline (135-150 NaCl) can be employed as the pharmaceutically acceptable carrier, but other suitable carriers will suffice. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc. These compositions can be sterilized using the techniques known in the art or, alternatively, they can be produced under sterile conditions. The resulting aqueous solutions may be packaged for use or filtered under aseptic conditions and lyophilized, the lyophilized preparation being combined with a sterile aqueous solution prior to administration.

In addition to the above-described formulations, the compositions may also be formulated as a depot preparation. Such long acting formulations may be administered by implantation (e.g., subcutaneously or intramuscularly) or by intramuscular injection. Thus, the active ingredients described herein can be formulated with suitable polymeric or hydrophobic materials (e.g., as an emulsion in an acceptable oil) or ion exchange resins, or as sparingly soluble derivatives (e.g., as a sparingly soluble salt).

### D. Doses

Generally, administered dosages will be effective to deliver picomolar to micromolar concentrations of each antagonist to the appropriate site or sites. However, one of ordinary skill in the art understands that the dose administered will vary depending on a number of factors, including, but not limited to, the particular combination of antagonists to be administered, the mode of administration, the type of application, the age of the patient, and the physical condition of the patient. Preferably, the smallest dose and concentration required to produce the desired result should be used. Dosage should be appropriately adjusted for children, the elderly, debilitated patients, and patients with cardiac anti/or liver disease. Further guidance can be obtained from studies known in the art using experimental animal models for evaluating dosage. However, the therapeutic effect produced by the combination of antagonists described herein permits a wider margin of safety since lower doses of each antagonist can be administered without compromising efficacy.

Typically, the compositions of the present invention will contain a 5-HT₃ receptor antagonist such as ondansetron or a pharmaceutically acceptable salt thereof in an amount of from about 0.1 to about 100 mg, alternatively from about 0.1 to about 50 mg, alternatively from about 0.1 to about 25 mg, or alternatively from about 0.1 to about 5.0 mg per administration. In certain instances, the 5-HT₃ receptor antagonist is formulated in a dosage form to provide a dose of from about 0.1 to about 50 μg/kg, alternatively from about 0.1 to about 25 μg,/kg, or alternatively about 0.5, 1, 2, 4, 8, or 16 μg/kg per administration. Preferably, the lowest practicable dose of 5-HT₃ receptor antagonist that is sufficient to treat the subject is administered. For example, ondansetron or a pharmaceutically acceptable salt thereof can be administered at a low dose of less than or equal to about 5.0 mg, e.g., about 0.1, 0.25, 0.5, 0.75, 1.0, 1.5, 2.0, 2.5, 3.0, 3.5, 4.0, 4.5, or 5.0 mg per administration. Alternatively, ondansetron or a pharmaceutically acceptable salt thereof can be administered at a high dose of greater than about 5.0 Mg, e.g., at least about 6.0, 7.0, 8.0, 9.0, 10, 12.5, 15, 20, or 25 mg per administration.

Similarly, the compositions of the present invention will contain a selective dopamine D₂ receptor antagonist such as olanzapine or a pharmaceutically acceptable salt thereof in an amount of from about 0.1 to about 100 mg, alternatively from about 0.1 to about 50 mg, alternatively from about 0.1 to about 25 mg, alternatively from about 0.1 to about 10 mg, or alternatively about 0.25, 0.5, 1.0, 2.0, 2.5, 5.0, 7.5, 10, or 12.5 mg per administration. Preferably, the lowest practicable dose of selective dopamine D₂ receptor antagonist that is sufficient to treat the subject is administered. For example, olanzapine or a pharmaceutically acceptable salt thereof can be administered at a low dose of less than or equal to about 5.0 mg, e.g., about 0.1, 0.25, 0.5, 0.75, 1.0, 1.5, 2.0, 2.5, 3.0, 3.5, 4.0, 4.5, or 5.0 mg per administration. Alternatively, olanzapine or a pharmaceutically acceptable salt thereof can be administered at a high dose of greater than about 5.0 mg, at least about 6.0, 7.0, 8.0, 9.0, 10, 12.5, 15, 20, or 25 mg per administration.

It will be understood that the appropriate effective dosage to be administered to a subject can be evaluated in an appropriate patient population that has been selected based on factors such as age, weight, the severity of the disease or condition, and/or the ability of a subject to metabolize each of the antagonists. Accordingly, effective amounts of each antagonist may be different for selected patient populations. For example, subjects with a diminished capacity to metabolize one or more of the antagonists (i.e., subjects 65 years of age and older) can be administered a portion of a dose that would be administered to a subject with a normal capacity to metabolize each of the antagonists (e.g., a half-tablet dose).

### E. Kits

The present invention also relates to pharmaceutical compositions in kit form. The kit will typically comprise one or more containers containing the compositions of the present invention (e.g., a foil packet, a bottle, a vial, or any other type of container). In addition, the kit usually includes instructions on the administration of the compositions. For example, the compositions may be presented in a pack or dispenser device that contains one or more unit dosage forms comprising the active ingredients. The pack or dispenser can include metal or plastic fashioned as a blister pack. As is true for any of the formulations described herein, the pack or dispenser device can be packaged and accompanied by instructions for use and optionally, paraphernalia for administration (e.g., should the formulation be an aerosol, a dispenser can be included).

In certain instances, it may be desirable to provide a memory aid on the kit, e.g., in the form of numbers that correspond with the days of the regimen which the dosage form so specified should be administered. Another example of such a memory aid is a calendar printed on the kit. Other variations of memory aids will be readily apparent to one skilled in the art, such as, for example, a mechanical counter which indicates the number of daily doses that has been dispensed, a microchip memory coupled with a liquid crystal readout, or an audible reminder signal which reads out the date that the last daily dose has been taken and/or reminds one when the next dose is to be taken, and the like.

### IV. EXAMPLES

The following examples are offered to illustrate, but not to limit, the claimed invention.

### Example 1

### Prolonged Release Tablet Containing 4.0 mg Ondansetron HCl

3.3% ondansetron, 91.6% lactose, 2.5% citric acid, and 2.1% hydroxypropyl methylcellulose (Pharmacoat 606; Shin-Etsu Chemical Co.; Japan) are mixed together, granulated with water, dried, and calibrated. The granulate is then mixed with 0.5% magnesium stearate and compressed to a mass of 120 mg per tablet using a rotary tableting machine. Tablets are coated in an Accelacota pan coater with a sufficient quantity of the following mixture to obtain the desired dissolution profile: 2.0% ethylcellulose (Ethocel; Dow Chemical Co.; Midland, Mich.), 0.4% diethyl phthalate, 2.0% hydroxypropyl methylcellulose (Pharmacoat 606), 47.8% isopropanol, and 47.8% dichloromethane.

### Example 2

### Immediate Release Tablet Containing 2.5 mg Olanzapine

Tablets dosed at 2.5 mg olanzapine and with a unitary mass of 120 mg can be manufactured according to the same method as Example 1, but having the following composition: 2.01% olanzapine, 82% lactose, 10.0% microcrystalline cellulose (Avicel; FMC Corp.; Philadelphia, Pa.), 2.1% hydroxypropyl methylcellulose (Pharmacoat 606), 3.2% sodium carboxymethylcellulose (Primojel; Avebe; Netherlands), and 0.6% magnesium stearate.

### Example 3

### Capsule Containing Immediate and Prolonged Release Tablets

A pharmaceutical dosage form containing a 2.5 mg olanzapine immediate release tablet according to Example 2 and a 4.0 mg ondansetron prolonged release tablet according to Example 1 can be prepared within gelatin capsules.

### Example 4

### Capsule Containing Immediate and Prolonged Release Pellets

Capsules comprising a mixture of immediate release pellets and coated prolonged release pellets can be prepared as follows:

1. A suspension comprising 100 g olanzapine, 175 g ondansetron, and 100 g povidone (Plasdone K29/32; BASF; Germany) in 670 g ethanol is prepared.

2. 750 g of this suspension is sprayed onto 1060 g of 16-18 mesh microgranules in a fluid bed drier.

3. A solution comprising 25 g of methacrylate copolymer Eudragit® RL100, 143 g of methacrylate copolymer Eudragit® RS100, and 18.7 g of ethyl citrate Eudrafex® as plasticizer, is prepared in 1180 g of a 60:40 mini isopropanol/acetone mixture.

4. Pellets comprising olanzapine and ondansetron are coated with this polymer mixture by spraying in a fluid bed dryer, the final amount of coating being 20% by mass of the uncoated pellet mass.

5. After maturation of the pellets at 35° C. for 24 hours, a mixture of these coated pellets and the uncoated pellets previously described is prepared in a 1:1 ratio by olanzapine:ondansetron content, and are filled into gelatin capsules to give a total amount of olanzapine and ondansetron content per capsule.

### Example 5

### Tablet Containing Prolonged Release Pellets Embedded within a Matrix

Tablets comprising 5.0 mg olanzapine coated prolonged release pellets within a fast-disintegrating matrix comprising 8.0 mg ondansetron can be prepared as follows:

1. Prolonged release coated pellets containing 5.0 mg olanzapine are manufactured as described in Example 4. The pellets are then spray-coated using the same method with a layer of 20% by weight of microcrystalline cellulose.

2. A granulate comprising 8.4% ondansetron, 20.0% lactose, 62.9% microcrystalline cellulose (Avicel), 3.0% hydroxypropyl methylcellulose (Pharmacoat 606), 5.0% crospovidone (Kollidon CL: BASE; Germany), and 0.7% magnesium stearate is then prepared by wet granulation.

The granulate is mixed with the coated pellets in a ratio of 3 parts granulate to 2 parts coated pellets and the mixture compressed into tablets.

### Example 6

### Tablet Containing Prolonged Release Pellets Embedded within a Matrix

Tablets comprising 0.5 mg risperidone coated prolonged release pellets within a fast-disintegrating matrix comprising 1.0 mg ondansetron can be prepared as follows:

1. Prolonged release coated pellets containing 0.5 mg risperidone are manufactured as described in Example 4. The pellets are then spray-coated using the same method. with a layer of 20% by weight of microcrystalline cellulose.

2. A granulate comprising 1.05% ondansetron, 27.4% lactose, 62.9% microcrystalline cellulose (Avicel), 3.0% hydroxypropyl methylcellulose (Pharmacoat 606), 5.0% crospovidone (Kollidon BASF; Germany), and 0.7% magnesium stearate is then prepared by wet granulation.

3. The granulate is mixed with the coated pellets in a ratio of 3 parts granulate to 2 parts coated pellets and the mixture compressed into tablets.

### Example 7

### Bilayer Immediate/Prolonged Release Tablet

Bilayer immediate/prolonged release tablets comprising 2.5 mg olanzapine and 4.0 mg ondansetron can be prepared as follows:

1. An immediate release granulate containing 2.2% olanzapine, 70.3% lactose 150 mesh, 20.0% microcrystalline cellulose, 2.5% hydroxypropyl methylcellulose (Pharmacoat 606), 3.8% sodium carboxymethylcellulose, and 1.0% magnesium stearate is prepared by the wet granulation process described in Example 1.

2. A prolonged release granulate containing 3% ondansetron, 43.0% lactose 150 mesh, 20.0% microcrystalline cellulose, 8.4% tartaric acid, 25.0% hydroxypropyl methylcellulose (Metolose 90SH4000; Shin-Etsu Chemical Co.; Japan), and 1.0% magnesium stearate is prepared by the wet granulation process described in Example

3. The mixtures are then compressed into bilayer tablets using an alternative tablet press. Each 250 mg tablet contains 125 mg of the immediate release granulate and 125 mg of the prolonged release granulate.

The in vitro dissolution profiles of the tablets may be established using the Apparatus 2 of the United States Pharmacopeia. Three dissolution media can be employed: 0.01 M hydrochloric acid, 0.025 M potassium phosphate buffer at pH 6.8, and 0.015 M potassium phosphate buffer at pH 7.5. The volume of dissolution medium can be 500 ml, maintained at 37±0.50° C. A grill can be placed in the bottom of each vessel to prevent sticking of the tablet to the glass surface. The percentage dissolved can be determined by measurement of the UV absorbance.

### Example 8

### Trilayer Immediate/Prolonged Release Tablet

Three-layer immediate/prolonged release tablets comprising 12.5 mg olanzapine can be prepared as follows:

1. An immediate release granulate (layer 1) containing 5.0% olanzapine, 67.7% lactose 150 mesh, 20.0% microcrystalline cellulose, 2.5% hydroxypropyl methylcellulose (Pharmacoat 606). 3.8% sodium carboxymethylcellulose, and 1.0% magnesium stearate is prepared by the wet granulation process described in Example 1.

2. A granulate without any active ingredient (layer 2) containing 60.0% lactose (spray dried), 24.0% microcrystalline cellulose, 10.0% tartaric acid, 5.0% hydroxyethylcellulose, and 1.0% magnesium stearate is prepared by the wet granulation process described in Example 1.

3. A prolonged release granulate (layer 3) containing 6.0% olanzapine. 40.0% lactose 150 mesh, 19.0% microcrystalline cellulose, 9.0% tartaric acid, 25.0% hydroxypropyl methylcellulose (Metolose 90SH4000), and 1.0% magnesium stearate is prepared by the wet granulation process described in Example 1.

4. The mixtures are then compressed as described in Example 6 into 3-layer tablets, with 100 mg of layer I containing 5.0 mg olanzapine, 100 mg of layer 2 (the middle layer), and 125 mg of layer 3 containing 7.5 mg olanzapine.

### Example 9

### Bilayer Immediate/Prolonged Release Tablet with Abuse Resistance-Conferring Substances

Coated bilayer immediate/prolonged release tablets comprising 10 mg olanzapine and containing an efferevescent couple and a dye in the immediate release layer can be prepared as follows:

1. A powder mixture for the immediate release layer is prepared by dry mixing 3.6% olanzapine, 11.3% anhydrous lactose, 24.3% microcrystalline cellulose, 5.0% povidone K30, 23.0% tartaric acid, 25.0% sodium bicarbonate, 3.0% sodium carboxymethylcellulose, and 0.8% Indigotine W6004. 2.0% sodium dodecyl sulfate, 1.0% colloidal silica, and 1.0% magnesium stearate are then added.

2. A prolonged release granulate is prepared by granulating 4.4% olanzapine, 36.0% lactose 150 mesh, 8.4% tartaric acid, 20.0% microcrystalline cellulose, and 30.0% hydroxypropyl methylcellulose (Metolose 90SH4000) with water. 0.2% colloidal silica and 1.0% magnesium stearate are mixed with the granulate after drying and sieving.

3. The mixtures are then compressed into bilayer tablets using a Manesty BL tablet press. Each tablet contains 10 mg olanzapine: the first immediate release layer with 12.5 mg of the powder mixture contains 4.5 mg olanzapine; and the prolonged release layer with 125 mg of the granulate mixture contains 5.5 mg olanzapine.

4. A film coating (4% with respect to the tablet mass) comprising 12% copovidone (Kollidon VA64; BASF; Germany), 12% ethylcellulose, 46% titanium dioxide, and 30% talc is applied as a 20% dispersion in absolute alcohol using a coating turbine (Glatt GC300).

The dissolution profile of the tablets is determined in 0.01M hydrochloric acid using the apparatus and method described in Example 6.

### Example 10

### Trilayer Immediate/Prolonged Release Tablet with Abuse Resistance-Conferring Substances

Coated trilayer immediate/prolonged release tablet comprising 2.5 mg olanzapine and 8.0 mg ondansetron and containing an effervescent couple and a dye can be prepared as follows:

1. A powder mixture for the immediate release layer is prepared by dry mixing 4.0% olanzapine, 36.4% microcrystalline cellulose, 5.0% povidone K30, 23.0% tartaric acid, 25.0% sodium bicarbonate, 3.0% sodium carboxymethylcellulose, 0.3% black iron oxide, and 0.8% Indigotine. 1.0% sodium dodecyl sulfate, 1.0% colloidal silica, and 0.5% magnesium stearate are then added.

3. A powder mixture for the anti-abuse layer is prepared by dry mixing 40.4% microcrystalline cellulose, 5.0% povidone K30, 23.0% tartaric acid, 25.0% sodium bicarbonate, 3.0% sodium carboxymethylcellulose, 0.3% black iron oxide, and 0.8% Indigotine. 1.0% sodium dodecyl sulfate, 1.0% colloidal silica, and 0.5% magnesium stearate are then added.

3. A prolonged release granulate is prepared by granulating 4.0% ondansetron, 36.0% lactose 150 mesh, 8.4% tartaric acid, 20.4% microcrystalline cellulose, and 30.0% hydroxypropyl methylcellulose (Metolose 90SH4000) with water. 0.2% colloidal silica and 1.0% magnesium stearate are mixed with the granulate after drying and sieving.

4. The mixtures are then compressed into trilayer table 5. The tablets are film coated as described in Example 8.

### Example 11

### Formulation and Fabrication of Transdermal Patches

This example illustrates the preparation of patches for the transdermal delivery of olanzapine and ondansetron.

A. Form illation

The starting solution may contain a mixture of olanzapine and ondansetron, the skin permeation enhancers dimethyl sulfoxide (DMSO), CERAPHYL® 31, ethyl lactate, and capric acid at a weight ratio of 4:1:1:0.8, the polyacrylate adhesive polymer DURO-TAK® 87-4098, and the plasticizer/humectant PVP/VA S-630. CERAPHYL® 31 (lauryl lactate) is manufactured by Van Dyk, a division of Mallinckrodt, Inc. (Belleville, N.J.). DURO-TAK® 87-4098 is available from National Starch and Chemical Co, (Bridgewater, N.J.). PVP/VA S-630 is available from ISP International Specialty Products, Inc. (Wayne, N.J.).

The amount of each component in the formulation may be determined by one skilled in the art to yield a finished, dried matrix composition suitable for use as a transdermal delivery agent. As a non-limiting example, the amount of the olanzapine and ondansetron can vary by plus or minus 5% w/w, the amount of PVP/VA 5-630 can vary from about 0% to about 10% w/w, the amount of the combination of skin permeation enhancers can vary from about 10% to about 60% w/w, and the amount of the DURO-TAK® 87-4098, which is the amount needed to reach a total of 100% for all ingredients, can range from about 30% to about 60% w/w.

### B. Fabrication Process

Transdermal delivery patches having the formulation described above can be fabricated as follows:

1. Olanzapine and ondansetron are weighed and put in a glass bottle.

2. The other excipients are added and the bottle is shaken by hand until the olanzapine, ondansetron, and PVP/VA-S630 are dissolved.

3. The DURO-TAK° 87-4098 (33% solid content) adhesive polymer solution is added and the bottle is sealed.

4. The contents of the bottle are stirred using a magnetic stirring bar at about 200 rpm at room temperature for 3 hours to form a homogeneous solution.

5. The bottle is allowed to stand for at least one hour or until all air bubbles disappear.

6. The resulting formulation is coated on a piece of backing laminate (Scotch Pak II 09; 3M Co.; St. Paul, Minn.) to a thickness of about 650 micrometers and subsequently dried at 60° C. for 15 minutes using a laboratory coating/drying machine (Model LTSV/LTH; Werner Mathis; Switzerland). After drying, the adhesive polymer matrix becomes approximately 100 micrometer thick.

7. The dried adhesive polymer matrix is laminated with a piece of release liner (Scotch Pak 1012; 3M Co.) of the same size to form a sheet. This sheet is cut into transdermal delivery patches of 10 cm² using a steel rule die and hydraulic press at 4000 psi. Each 10 cm² patch is individually packaged in a paper/foil pouch and stored in the refrigerator at 4° C.

The sheet can also be cut to form discs with any desired shape and size using a steel rule die and a hydraulic press. For example, the discs can be from about 5 to 100 cm², alternatively from about 8 to about 80 cm², or alternatively from about 10 to about 60 cm². A disc of 10 cm² is preferred because of its relatively small size, yet being capable of dispersing high levels of both antagonists. The shape of the discs can vary, e.g., they can be circular, square, rectangular, or any other desired shape. The resulting transdermal delivery system unit dosage forms are then placed in appropriate packaging for storage, such as paper and/or foil pouches, until they are to be applied in transdermal treatment.

### Dosing Schedules of Various Combinations

Due to simultaneous down-regulation of dopamine, combination with HT₃-antagonists enables the invention to reduce the dose of atypical antipsychotic (e.g., selective D2 receptor antagonist) for the treatment of alcohol-dependence. TABLE 1 compares doses of atypical antipsychotic agents for treatment of schizophrenia, which is administered as a monotherapy, and alcohol dependence, which is administered as a combination therapy with an HT₃-antagonist (e.g., 0.65 mg ondansetron). The dose of the atypical antipsychotic can be about 50% or less, alternatively about 45% or less, alternatively about 40% or less, alternatively about 35% or less, alternatively about 30% or less, alternatively about 25% or less of the dose used for single-drug treatment of delusional conditions such as schizophrenia.

TABLE 1

| Atypical antipsychotic | Schizophrenia dose | Alcohol dependence dose |
|---|---|---|
| agent | (monotherapy) | (combination therapy) |
| Aripiprazole | 15 mg | 7.5 mg |
| Olanzapine | 10 mg | 5 mg |
| Risperidone | 1 mg | 0.5 mg |
| Quetiapine | 200 mg | 100 mg |
| Ziprasidone | 50 mg | 25 mg |
| Clozapine | 60 mg | 30 mg |

TABLE 2 compares the doses for typical HT₃-antagonist as an anti-emetic, which is used in a monotherapy, to a dose for treatment of non-emetic conditions (e.g., alcohol-dependence), in which the HT₃-antagonist is administered in combination with an atypical antipsychotic, such as 5 mg of olanzapine. The doses of the HT₃-antagonist administered in the combination therapy can be about 30% or less, alternatively about 25% or less, alternatively about 10% or less, alternatively about 5% or less, alternatively about 1% or less, alternatively about 0.5% or less, alternatively about 0.25% or less alternatively about 0.20% or less of the dose used for single-drug treatment.

TABLE 2

| | Anti-emetic dose | Alcohol dependence dose |
|---|---|---|
| HT₃-antagonist | (monotherapy) | (combination therapy) |
| Ondansetron | 24 mg | 0.650 mg |
| Granisteron | 1 mg | 0.25 mg |
| Dolasetron | 100 mg | 25 mg |
| Palonosetron | 1 mg | 0.01 mg |

### Alcohol Dependence Study Protocol

The following protocol is offered to illustrate an alcohol dependence study in which patients are administered a combination therapy that includes a 5-HT₃ receptor antagonist (e.g., ondansetron) and a selective dopamine D₂ or D₂-like receptor antagonist (e.g., olanzapine).

1. About 300 (70% male) patients who meet the Diagnostic and Statistical Manual of Mental Disorders for alcohol dependence are enrolled in the study. The enrollers are typically 25 to 65 years old with Alcohol Use Disorders Inventory Test (AUDIT) score of 8 or more, and patients consuming greater than or equal to (≧) 5 drinks a day if the enroller is a man, and greater than or equal to (≧) 4 drinks if the enrollee is a woman.

2. Self-reported number of heavy drinking days as determined by Timeline Follow-back (TUB) interview process is the primary efficacy variable for the study.

3. The patients undergo urine toxicology screen for narcotics, amphetamines, or sedatives-hypnotics at the time of dosing.

4. Although abstinence is not the criterion for eligibility for the study, desire to stop drinking is often the treatment goal for patients.

5. Patients are not included in the trial if they have co-morbid psychiatry conditions or are found to abuse any other substance other than alcohol.

6. Patients are not included in the trial if they are taking medications that can potentially interact with alcohol.

7. At enrollment (Visit 0), after providing written, informed consent, patients undergo assessment of:
Physical health, which includes physical examination, vital signs measurement, and laboratory tests that includes blood alcohol concentration measurement.
Psychiatric diagnosis for absence of co-morbid conditions
Age of onset and their drinking profiles, which includes number of drinks per day on the basis of the TUT procedure.

8. Eligible patients are invited back for next visit (Visit 1) in which they are randomized either to receive the placebo or the active agent.

9. At Visit 1, patients receive their first cognitive behavioral therapy (CBT) and instructions for twice-daily dosing.

10. Patients are also instructed to return to the clinic on a weekly basis for another 8 weeks.

11. At Visit 2 (after 1 week of receiving single-blind placebo), patients receive their second (CBT) and undergo TLFB interview for drinking and drinking profile.

12. At Visit 9 (last day of the treatment), in addition to TLFB interviews, patients undergo a final battery of physical, psychiatric, and laboratory tests to establish their health status.

### Various Combination Therapy Doses

TABLE 3 contains various dosages for different 5HT₃ receptor antagonists administered in combination with D₂ or D₂-like receptor antagonists, such as atypical antipsychotics.

TABLE 3

| Example | 5 HT₃ Receptor Antagonists (mg) | | | | D₂ or D₂~like Receptor Antagonists (atypical antipsychotics) (mg) | | | | | |
|---|---|---|---|---|---|---|---|---|---|---|
| No. | Ondansetron | Granisteron | Dolasetron | Palonosetron | Aripiprazole | Olanzapine | Risperidone | Quetiapine | Ziprasidone | Clozapine |
| 1 | 0.325 | | | | | 1 | | | | |
| 2 | 0.65 | | | | 7.5 | | | | | |
| 3 | | 0.25 | | | 7.5 | | | | | |
| 4 | | | 25 | | 7.5 | | | | | |
| 5 | | | | 0.01 | 7.5 | | | | | |
| 6 | 0.65 | | | | | 5.0 | | | | |
| 7 | | 0.25 | | | | 5.0 | | | | |
| 8 | | | 25 | | | 5.0 | | | | |
| 9 | | | | 0.01 | | 5.0 | | | | |
| 10 | 0.65 | | | | | | 0.5 | | | |
| 11 | | 0.25 | | | | | 0.5 | | | |
| 12 | | | 25 | | | | 0.5 | | | |
| 13 | | | | 0.01 | | | 0.5 | | | |
| 14 | 0.65 | | | | | | | 100 | | |
| 15 | | 0.25 | | | | | | 100 | | |
| 16 | | | 25 | | | | | 100 | | |
| 17 | | | | 0.01 | | | | 100 | | |
| 18 | 0.65 | | | | | | | | 50 | |
| 19 | | 0.25 | | | | | | | 50 | |
| 20 | | | 25 | | | | | | 50 | |
| 21 | | | | 0.01 | | | | | 50 | |
| 22 | 0.65 | | | | | | | | | 30 |
| 23 | | 0.25 | | | | | | | | 30 |
| 24 | | | 25 | | | | | | | 30 |
| 25 | | | | 0.01 | | | | | | 30 |

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method for treating obsessive, impulsive, and compulsive behavioral activities in a patient, said method comprising the steps of:
providing a pharmaceutical composition comprising about 0.2 to about 8.0 mg of ondansetron and about 0.1 to about 0.5 mg of risperidone; and
administering the composition to the patient.

2. The method of claim 1, wherein the pharmaceutical composition comprises about 0.2 to about 0.8 mg of ondansetron.

3. The method of claim 1, wherein the pharmaceutical composition comprises about 0.325 mg of ondansetron.

4. The method of claim 1, wherein the pharmaceutical composition comprises about 0.65 mg of ondansetron.

5. The method of claim 1, wherein the pharmaceutical composition comprises about 0.5 mg of risperidone.

6. The method of claim 1, wherein the obsessive, impulsive, and compulsive behavioral activities include substance abuse or substance dependence.

7. A method for treating obsessive, impulsive, and compulsive behavioral activities in a patient, said method comprising the steps of:
providing a pharmaceutical composition comprising about 0.2 to about 8.0 mg of ondansetron and a therapeutically effective amount of risperidone; and
administering the composition to the patient daily.

8. The method of claim 7, wherein the therapeutically effective amount of risperidone is about 0.1 to about 0.5 mg.

9. The method of claim 7, wherein the composition is administered daily.

10. The method of claim 7, wherein the composition is administered twice daily.

11. A method for treating obsessive, impulsive, and compulsive behavioral activities in a patient, said method comprising the steps of:
providing a pharmaceutical composition comprising a therapeutically effective amount of ondansetron and a therapeutically effective amount of risperidone; and
administering the composition to the patient.

12. The method of claim 11, wherein the pharmaceutical composition comprises about 0.2 to about 0.8 mg of ondansetron.

13. The method of claim 11, wherein the pharmaceutical composition comprises about 0.325 mg of ondansetron.

14. The method of claim 11, wherein the pharmaceutical composition comprises about 0.65 mg of ondansetron.

15. The method of claim 11, wherein the pharmaceutical composition comprises about 0.5 mg of risperidone.

16. The method of claim 11, wherein the obsessive, impulsive, and compulsive behavioral activities include substance abuse or substance dependence.
